(19) United States
(12) Patent Application Publication
(10) Pub. No.: US 2026/0007745 A1
(43) Pub. Date: Jan. 8, 2026

(54) ALDH Inhibitors to Promote Immune Cell Expansion

(71) Applicant: University of Pittsburgh - Of the Commonwealth System of Higher Education, Pittsburgh, PA (US)

(72) Inventors: Ronald J. Buckanovich, Pittsburgh, PA (US); Anda Mioara Vlad, Wexford, PA (US)

(21) Appl. No.: 18/698,968

(22) PCT Filed: Oct. 7, 2022
(86) PCT No.: PCT/US2022/046056

§ 371 Date: Apr. 5, 2024

Related U.S. Application Data
(60) Provisional application No. 63/253,777, filed on Oct. 8, 2021.

(51) Int. Cl. A61K 40/11; A61K 31/135; A61K 31/4035; A61K 35/15; A61K 35/17; A61K 39/00; A61K 40/19; A61K 40/24; A61K 40/31; A61P 35/00; C07K 16/28; C12N 5/077; C12N 5/0784

(52) U.S. Cl. CPC A61K 40/11; A61K 31/135; A61K 31/4035; A61K 35/15; A61K 35/17; A61K 39/0011; A61K 40/19; A61K 40/24; A61K 40/31; A61P 35/00; C07K 16/2896; C12N 5/0639; C12N 5/0669; C12N 2501/999; C12N 2506/1353

(57) ABSTRACT

Using an aldehyde dehydrogenase-1A enzyme inhibitor (ALDHi) for expanding bone marrow cells and/or dendritic cells and for adjuvant therapy to an immunotherapy

### CROSS REFERENCE TO RELATED APPLICATIONS

This application claims priority to U.S. Provisional Patent Application No. 63/253,777 filed Oct. 8, 2021, the disclosure of which is incorporated by reference in its entirety.

### STATEMENT REGARDING FEDERALLY SPONSORED RESEARCH

This invention was made with government support under Grant No. CA238315, awarded by the National Institutes of Health. The government has certain rights in the invention.

Provided herein are adjuvant therapies for cancers, as well as methods and compositions useful for expansion of bone marrow cell and/or dendritic cell populations.

Immunotherapy for cancer has shown great promise, however the majority of patients still do not gain a benefit. Generation of consistently robust immune response and cell populations has not been achieved. Cancer immunotherapies include: cell-based immunotherapies, immunomodulators such as checkpoint inhibitors, cancer vaccines, antibody-based targeted therapies, and oncolytic viruses. For example, cell-based immunotherapies, also referred to as adoptive cell therapy, uses cells of the immune system to target cancer cells. Various immunotherapies rely on the effective elicitation of an appropriate immune response, including a proinflammatory/antitumor immune response, and favoring cytotoxic CD8⁺ T cells (T_{C}), DC, and CD4⁺ T_{Helper} (T_{H}) cells, while disfavoring T_{Reg} cells. Further, T_{Reg}s are seen to inhibit antitumor immunity in many cancer patients and correlate with poor clinical outcome.

In CAR-T therapies, therapeutic CAR-T cells are T cells modified with a chimeric antigen receptor (CAR) that targets an antigen expressed on tumor cells, but not on normal cells in a patient (see, e.g., Mohanty, R., et al. (2019). CAR T cell therapy: A new era for cancer treatment (Review). Oncology Reports, 42, 2183-2195). Chimeric antigen receptors are engineered to redirect lymphocytes to recognize and eliminate cells expressing a specific target antigen. Chimeric antigen receptor binding to target antigens is independent of the MHC receptor, resulting in vigorous T cell activation and powerful anti-tumor responses. One of the limitations of CAR-T therapies is the immunosuppressive microenvironment associated with malignancies (see, generally, Sterner, R. C., et al. CAR-T cell therapy: current limitations and potential strategies. Blood Cancer J. 11, 69 (2021)).

Dendritic cell therapies, such as DC-based anti-tumor vaccines, are an approach to enhancement of targeted immune response, e.g., against cancer cells (see, generally, Calmeiro J, et al. Dendritic Cell Vaccines for Cancer Immunotherapy: The Role of Human Conventional Type 1 Dendritic Cells. Pharmaceutics. 2020; 12(2):158). Dendritic cells are antigen-presenting cells (APCs) that derive from, and therefore can be derived from, bone-marrow cells.

Another form of cancer treatment is the use of T cell-targeted immunomodulators that block immune checkpoints like CTLA-4, PD1 or PDL1. Ipilimumab, was the first approved antibody blocking an immune checkpoint (CTLA4), and approval of PD1-targeting antibodies (pembrolizumab and nivolumab) and PDL1-targeting antibodies (atezolizumab and durvalumab) followed. Anti-PD1/PDL1 antibodies are widely prescribed anticancer therapies. T-cell-targeted immunomodulators are now used alone, or (when single agent efficacy is low) in combination with other drugs and/or chemotherapeutics for treatment of about 50 cancer types, with numerous clinical trials pending (see, e.g., Robert, C. A decade of immune-checkpoint inhibitors in cancer therapy. Nat Commun 11, 3801 (2020)). Cancer cells can suppress an effective immune response to tumor antigens, a mechanism called immune evasion (See, e.g., Marin-Acevedo, J. A., Kimbrough, E. O. & Lou, Y. Next generation of immune checkpoint inhibitors and beyond. J Hematol Oncol 14, 45 (2021)).

Methods and compositions useful as adjuncts (adjuvants) to improve immunotherapies, e.g., cancer immunotherapies, and to reduce immunosuppression are needed.

### SUMMARY

Aldehyde dehydrogenase-1A enzymes (ALDH1A) represent one novel target cancer immunotherapy. ALDH1A enzymes mediate the biosynthesis of retinoic acid (RA) to regulate numerous cellular processes. A number of novel ALDH1A family inhibitors (ALDHis) have recently been identified. Our data indicate that ALDHi treatment of T cells is associated with enhanced differentiation of and proliferative response of CD8⁺ T cells, with a suppression of CD4⁺ T regulatory cells. Furthermore, ALDHis are seen to promote dendritic cell (DC) proliferation and can be used to significantly increase DC numbers for DC vaccine based therapy ALDHis are, therefore, able to create a proinflammatory/antitumor immune response and support immunotherapeutics. For example, when used in vivo in conjunction with a tumor vaccine, an ALDHi is seen to increase tumor control.

Provided herein is a method of expanding bone marrow cells and/or dendritic cell populations, comprising culturing bone-marrow cells and/or dendritic cells in culture medium comprising an amount of an Aldehyde dehydrogenase-1A enzyme inhibitor (ALDHi) effective to increase a population of the bone-marrow cells and/or dendritic cells as compared to an increase in the population of the bone-marrow cells and/or dendritic cells grown in the same culture medium and conditions without the ALDHi in the culture medium.

Also provided herein is an immunotherapy method for increasing an immune response to a pathogen or cancer cell antigen in a patient, comprising: administering to a patient an immunotherapeutic agent for increasing an antigen-specific immune response in a patient; and administering to the patient an adjuvant therapy to the administration of the immune cell, an amount of an aldehyde dehydrogenase-1A enzyme inhibitor (ALDHi) effective to reduce T_{REG} activity in the patient.

The following numbered clauses describe various aspects and/or embodiments of the present invention.

Clause 1. A method of expanding bone marrow cells and/or dendritic cell populations, comprising culturing bone-marrow cells and/or dendritic cells in culture medium comprising an amount of an Aldehyde dehydrogenase-1A enzyme inhibitor (ALDHi) effective to increase a population of the bone-marrow cells and/or dendritic cells as compared to an increase in the population of the bone-marrow cells and/or dendritic cells grown in the same culture medium and conditions without the ALDHi in the culture medium.

Clause 2. The method of clause 1, wherein the ALDHi has the structure:

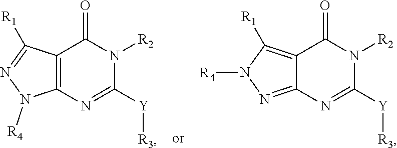

including pharmaceutically acceptable salts, solvates, and/or prodrugs thereof, wherein R₁, R₂, R₃, R₄, or Y independently can include any chemical moiety that permits the resulting compound to inhibit ALDH activity.

Clause 3. The method of clause 2, wherein R₁, R₂, R₃, R₄, or Y, independently, are any chemical moiety as described herein, and wherein Y may be present or absent.

Clause 4. The method of clause 2 or 3, wherein R₁ is H or C₁-C₃ alkyl (substituted or non-substituted).

Clause 5. The method of any one of clauses 2-4, wherein R₂ is H, C₀-C₃-alkyl-aryl or C₀-C₃-alkyl-heteroaryl, wherein the alkyls can be independently substituted or non-substituted, such as CH₃, or selected from one of the following:

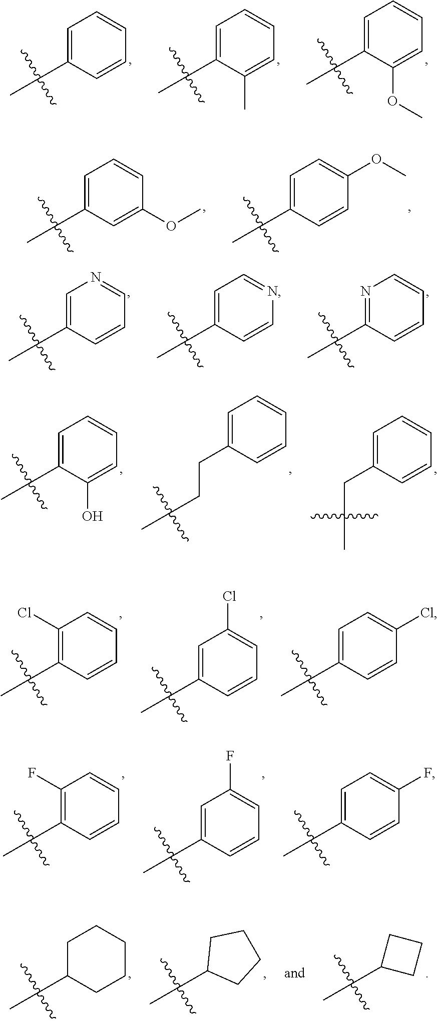

Clause 6. The method of any one of clauses 2-5, wherein Y is a linker, and optionally an alkyl, alkenyl, heteroalkyl, or heteroalkenyl, where the hetero atom is S, N, or O that is optionally substituted, such as a linker shown herein.

Clause 7. The method of any one of clauses 2-6, wherein R₃ is H, C₀-C₈ alkyl, C₀-C₂-alkyl-aryl or C₀-C₂-alkyl-heteroaryl, wherein the alkyls are independently substituted or non-substituted, such as a moiety as shown herein for R₃.

Clause 8. The method of any one of clauses 2-7, wherein R₄ is H, C₁-C₆ alkyl or cycloalkyl, optionally substituted, wherein one or more carbons may be replaced by oxygen, and wherein the alkyls are independently substituted or non-substituted, such as a structure of one of the following:

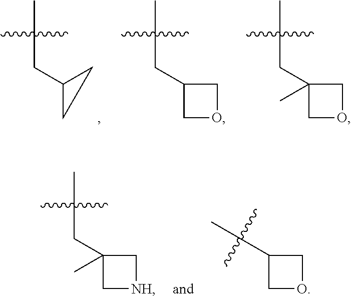

Clause 9. The method of clause 1, wherein the ALDHi is selected from one of the following, or a pharmaceutically acceptable salt thereof: 6-((3-fluorobenzyl)thio)-5-(o-tolyl)-1H-pyrazolo[3,4-d]pyrimidin-4(5H)-one, 6-((3-fluorobenzyl)thio)-5-phenyl-1H-pyrazolo[3,4-d]pyrimidin-4(5H)-one, 6-((3-fluorobenzyl)thio)-1-methyl-5-(o-tolyl)-1H-pyrazolo[3,4-d]pyrimidin-4(5H)-one, 6-((3-fluorobenzyl)thio)-2-methyl-5-(o-tolyl)-2H-pyrazolo[3,4-d]pyrimidin-4(5H)-one, 1-ehyl-6-((3-fluorobenzyl)thio)-5-(o-tolyl)-1H-pyrazolo[3,4-d]pyrimidin-4(5H)-one, 2-ethyl-6-((3-fluorobenzyl)thio)-5-(o-tolyl)-2H-pyrazolo[3,4-d]pyrimidin-4(5H)-one, 1-(cyclopropylmethyl)-6-((3-fluorobenzyl)thio)-5-(o-tolyl)-1H-pyrazolo[3,4-d]pyrimidin-4(5H)-one, 2-(cyclopropylmethyl)-6-((3-fluorobenzyl)thio)-5-(o-tolyl)-2H-pyrazolo[3,4-d]pyrimidin-4(5H)-one, 2-((3-fluorobenzyl)thio)-1-(oxetan-3-yl)-5-(o-tolyl)-1H-pyrazolo[3,4-d]pyrimidin-4(5H)-one, 6-((3-fluorobenzyl)thio)-2-(oxetan-3-yl)-5-(o-tolyl)-2H-pyrazolo[3,4-d]pyrimidin-4(5H)-one, 6-(3-fluorobenzyl)thio)-1-(oxetan-3-ylmethyl)-5-(o-tolyl)-1H-pyrazolol[3,4-d]pyrimidin-4(5H)-one, 6-((3-fluorobenzyl)thio)-2-(oxetan-3-ylmethyl)-5-(o-tolyl)-2H-pyrazolo[3,4-d]pyrimidin-4(5H)-ore, 6-((3-fluorobenzyl)thio)-2-methyl-5-phenyl-2H-pyrazolo[3,4-d]pyrimidin-4(5H)-one, 1-ethyl-6-((3-fluorobenzyl)thio)-5-phenyl-1H-pyrazolo[3,4-d]pyrimidin-4(5H)-one, 2-ethyl-6-((3-fluorobenzyl)thio)-5-phenyl-2H-pyrazolo[3,4-d]pyrimidin-4(5H)-one, 1-(cyclopropylmethyl)-6-((3-fluorobenzyl)thio)-5-phenyl-1H-pyrazolo[3,4-d]pyrimidin-4(5H)-one, 2-(cyclopropylethyl)-6-((3-fluorobenzyl)thio)-5-phenyl-2H-pyrazolo[3,4-d]pyrimidin-4(5H)-one, 6-((3-fluorobenzyl)thio)-1-(oxetan-3-yl)-5-phenyl-1H-pyrazolo[3,4-d]pyrimidin-4(5H)-one, 6-((3-fluorobenzyl)thio)-2-(oxetan-3-yl)-5-phenyl-2H-pyrazolo[3,4-d]pyrimidin-4(5H)-one, 6-((3-fluorobenzyl)thio)-1-(oxetan-3-ylmethyl)-5-phenyl-1H-pyrazolo[3,4-d]pyrimidin-4(5H)-one, 6-((3-fluorobenzyl)thio)-2-(oxetan-3-ylmethyl-5-phenyl-2H-pyrazolo[3,4-d]pyrimidin-4(5H)-one, 6-((3 fluorobenzyl)amino)-5-(o-tolyl)-1H-pyrazolo[3,4-d]pyrimidin-4(5H)-one, 6-((3 fluorophenethyl)amino)-1-methyl-5-(o-tolyl)-1H-pyrazolo[3,4-d]pyrimidin-4(5H)-one, 6-((3 fluorobenzyl)oxy)-1-methyl-5-(o-tolyl)-1H-pyrazolo[3,4-d]pyrimidin-4(5H)-one, 6-((3 fluorophenoxy)methyl)-1-methyl-5-phenyl-1H-pyrazolo[3,4-d]pyrimidin-4(5H)-one, 6-(((3 fluorobenzyl)amino)methyl)-1-methyl-5-phenyl-1H-pyrazolo[3,4-d]pyrimidin-4(5H)-one, 6-(3-fluorobenzyl)thio)-1-methyl-5-phenyl-1H-pyrazolo[3,4-d]pyrimidin-4(5H)-one, 6-((3-fluorobenzyl)thio)-5-(2-methoxyphenyl)-1-methyl-1H-pyrazolo[3,4-d]pyrimidin-4(5H)-one. 6-((3-fluorobenzyl)thio)-5-(3-methoxyphenyl)-1-methyl-1H-pyrazolo[3,4-d]pyrimidin-4(5H)-one, 6-((3-fluorobenzyl)thio)-5-(4-methoxyphenyl)-1-methyl-1H-pyrazolo[3,4-d]pyrimidin-4(5H)-one 6-((3-fluorobenzyl)thio)-1-methyl-5-(pyridin-3-yl)-1H-pyrazolo[3,4-d]pyrimidin-4(5H)-one, 6-((3-fluorobenzyl)thio)-1-methyl-5-(pyridin-4-yl)-1H-pyrazolo[3,4-d]pyrimidin-4(5H)-one, 6-((3-fluorobenzyl)thio)-5-(2-hydroxyphenyl)-1-methyl-1H-pyrazolo[3,4-d]pyrimidin-4(5H)-one, 6-((3-fluorobenzyl)thio)-1-methyl-5-(pyridin-2-yl)-1H-pyrazolo[3,4-d]pyrimidin-4(5H)-one, 6-((3-fluorobenzyl)thio)-1-methyl-5-phenethyl-1H-pyrazolo[3,4-d]pyrimidin-4(5H)-one, 5-benzyl-6-((3-fluorobenzyl)thio)-1-methyl-1H-pyrazolo[3,4-d]pyrimidin-4(5H)-one, 6-((3 fluorobenzyl)thio)-1,5-dimethyl-1H-pyrazolo[3,4-d]pyrimidin-4(5H)-one, 6-(isopentylthio-1-methyl-5-phenyl-1H-pyrazolo[3,4-d]pyrimidin-4(5H)-one, 6-((3-fluorobenzyl)thio)-3-methyl-5-(o-tolyl)-2H-pyrazolo[3,4-d]pyrimidin-4(5H)-one, 6-(benzo[b]thiophen-2-ylmethoxy)-1-methyl-5-(o-tolyl-1H-pyrazolo[3,4-d]pyrimidin-4(5H)-one, 5-(2-chlorophenyl)-6-(3-fluorobenzylthio)-1-methyl-1H-pyrazolo[3,4-d]pyrimidin-4(5H)-one, 5-(2-chlorophenyl)-6-((3-fluorobenzyl)thio)-1-methyl-1H-pyrazolo[3,4-d]pyrimidin-4(5H)-one, 5-(3-chlorophenyl)-6-((3-fluorobenzyl)thio) 1-methyl-1H-pyrazolo[3,4-d]pyrimidin-4(5H)-one, 5-(4-chlorophenyl)-6-((3-fluorobenzyl)thio) 1-methyl-1H-pyrazolo[3,4-d]pyrimidin-4(5H)-one, 6-((3-fluorobenzyl)thio)-5-(2-fluorophenyl)-1-methyl-1H-pyrazolo[3,4-d]pyrimidin-4(5H)-one, 5-cyclohexyl-6-((3-fluorobenzyl)thio)-1 methyl-1H-pyrazolo[3,4-d]pyrimidin-4(5H)-one, 5-cyclopentyl-6-((3-fluorobenzyl)thio)-1 methyl-1H-pyrazolo[3,4-d]pyrimidin-4(5H)-one, 5-cyclobutyl-6-((3-fluorobenzyl)thio)-1 methyl-1H-pyrazolo[3,4-d]pyrimidin-4(5H)-one, (E)-6-(3-fluorostyryl)-1-methyl-5-phenyl-1H-pyrazolo[3,4-d]pyrimidin-4(5H)-one, 6-(3-fluorophenethyl)-1-methyl-5-phenyl-1H-pyrazolo[3,4-d]pyrimidin-4(5H)-one, 6-(((3-fluorophenyl)amino)methyl)-1-methyl-5-phenyl-1H-pyrazolo[3,4-d]pyrimidin-4(5H)-one, 6-(((3-fluorobenzyl)oxy)methyl-1-methyl-5-phenyl-1H-pyrazolo[3,4-d]pyrimidin-4(5H)-one, 6-(((3-fluorophenyl)thio)methyl)-1-methyl-5-phenyl-1H-pyrazolo[3,4-d]pyrimidin-4(5H)-one, 1-methyl-6-((2-oxo-2-phenylethyl)thio)-5-phenyl-1H-pyrazolo[3,4-d]pyrimidin-4(5H)-one, 3-(((1-methyl-4-oxo-5-phenyl-4,5-dihydro-1H pyrazolo[3,4-d]pyrimidin-6-yl)thio)methyl)benzonitrile, 6-((3-methoxybenzyl)thio)-1-methyl-5-phenyl-1H-pyrazolo[3,4-d]pyrimidin-4(5H)-one, 6-(benzylthio)-1-methyl-5-phenyl-1H-pyrazolo[3,4-d]pyrimidin-4(5H)-one, 1-methyl-5-phenyl-6-((3-(2,2,2-trifluoroacetyl)benzyl)thio)-1H-pyrazolo[3,4-d]pyrimidin-4(5H)-one, 3-(((1-methyl-4-oxo-5-phenyl-4,5dihydro-1H-pyrazolo[3,4-d]pyrimidin-6-yl)thio)methyl)benzaldehyde, 4-(((1-methyl-4-oxo-5-phenyl-4,5-dihydro-1H-pyrazolo[3,4-d]pyrimidin-6-yl)thio)methyl)benzaldehyde, 6-((3-fluorobenzyl)oxy)-1-methyl-5-phenyl-1H-pyrazolo[3,4-d]pyrimidin-4(5H)-one, 1-methyl-5-phenyl-6-((1-phenylethyl)thio)-1H-pyrazolo[3,4-d]pyrimidin-4(5H)-one, 6-(((3-fluorophenyl)(methyl)amino)methyl)-1-methyl-5-phenyl-1H-pyrazolo[3,4-d]pyrimidin-4(5H)-one, 6-((3-fluorobenzyl)oxy)-2-(oxetan-3-ylmethyl)-5-phenyl-2H-pyrazolo[3,4-d]pyrimidin-4(5H)-one, 2-(oxetan-3-ylmethyl)-5-phenyl-6-((1-phenylethyl)thio)-2H-pyrazolo[3,4-d]pyrimidin-4(5H)-one, (R)-1-methyl-5-phenyl-6-((1-phenylethyl)thio)-1H-pyrazolo[3,4-d]pyrimidin-4(5H)-one, (S)-1-methyl-5-phenyl-6-((1-phenylethyl)thio)-1H-pyrazolo[3,4-d]pyrimidin-4(5H)-one, 1-methyl-5-phenyl-6-(1-phenylethoxy)-1H-pyrazolo[3,4-d]pyrimidin-4(5H)-one, 6-((3-fluorobenzyl)thio)-2-((3-methyloxetan-3-yl)methyl)-5-phenyl-2H-pyrazolo[3,4-d]pyrimidin-4(5H)-one, 3-((6-(3-fluorobenzyl)thio)-4-oxo-5-phenyl-4,5-dihydro-2H-pyrazolo[3,4-d]pyrimidin-2-yl)methyl)azetidin-1-ium sulfate, 1-methyl-5-phenyl-6-((1-phenylpropyl)thio)-1H-pyrazolo[3,4-d]pyrimidin-4(5H)-one, 6-((1(3-fluorophenyl)ethyl)thio)-1-methyl-5-phenyl-1H-pyrazolo[3,4-d]pyrimidin-4(5H)-one, 6-(cyclohexylthio)-1-methyl-5-phenyl-1H-pyrazolo[3,4-d]pyrimidin-4(5H)-one, 1-(oxetan-3-yl)-5-phenyl-6-((1-phenylethyl)thio)-1H-pyrazolo[3,4-d]pyrimidin-4(5H)-one, 2-(oxetan-3-yl)-5-phenyl-6-((1-phenylethyl)thio)-2H-pyrazolo[3,4-d]pyrimidin-4(5H)-one, 1-methyl-6-((2-oxocyclopentyl)thio)-5-phenyl-1H-pyrazolo[3,4-d]pyrimidin-4(5H)-one, 1-methyl-6-((2-oxocyclohexyl)thio)-5-phenyl-1H-pyrazolo[3,4-d]pyrimidin-4(5H)-one, 1-methyl-5-phenyl-6-((2-phenylpropan-2-yl)thio)-1H-pyrazolo[3,4-d]pyrimidin-4(5H)-one, 6-((1-(3-hydroxyphenyl)ethyl)thio)-1-methyl-5-phenyl-1H-pyrazolo[3,4-d]pyrimidin-4(5H)-one, 1-methyl-5-phenyl-6-((1-(pyridin-2-yl)ethyl)thio)-1H-pyrazolo[3,4-d]pyrimidin-4(5H)-one, and 1-methyl-5-phenyl-6-((1-pyridin-3-yl)ethyl)thio)-1H-pyrazolo[3,4-d]pyrimidin-4(5H)-one.

Clause 10. The method clause 1, wherein the ALDHi is selected from a structure as depicted in FIGS. 2A-2C, or a pharmaceutically acceptable salt thereof:

Clause 11. The method of clause 1, wherein the ALDHi is selected from 673A, 257913 (913), 258085 (085), 262548 (548), 263052 (052), 263117 (117), 263118 (118), 263119 (119), 259122 (122), 263646 (646), 264202 (202), or a pharmaceutically acceptable salt thereof:

Clause 12. The method of clause 1, wherein the ALDHi is 673A, or a pharmaceutically acceptable salt thereof.

Clause 13. The method of clause 1, wherein the ALDHi has the structure:

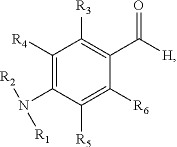

wherein R₁ and R₂ are independently or together, H, an alkyl, a cycloalkyl, together form a cycloalkyl or heterocyclic alkyl or aryl, an aryl, an alkenyl, a cycloalkyl, alkynl, or a substituted version of the aforementioned groups and R₃-R₆ are independently a halogen, H, an alkyl, a cycloalkyl, an aryl, an alkenyl, a cycloalkyl, alkynl, or a substituted version of the aforementioned groups, or a pharmaceutically acceptable salt thereof.

Clause 14. The method of clause 13, wherein the ALDHi has the structure:

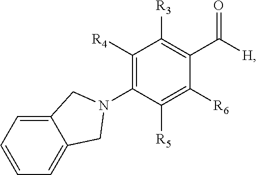

wherein and R₃-R₆ are independently a halogen, H, an alkyl, a cycloalkyl, an aryl, an alkenyl, a cycloalkyl, alkynl, or a substituted version of the aforementioned groups, or a pharmaceutically acceptable salt thereof.

Clause 15. The method of clause 1, wherein the ALDHi has a structure selected from:

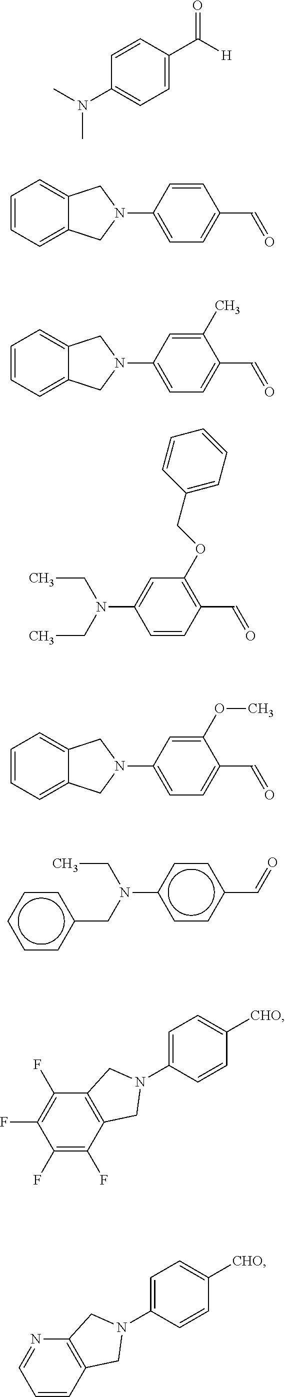

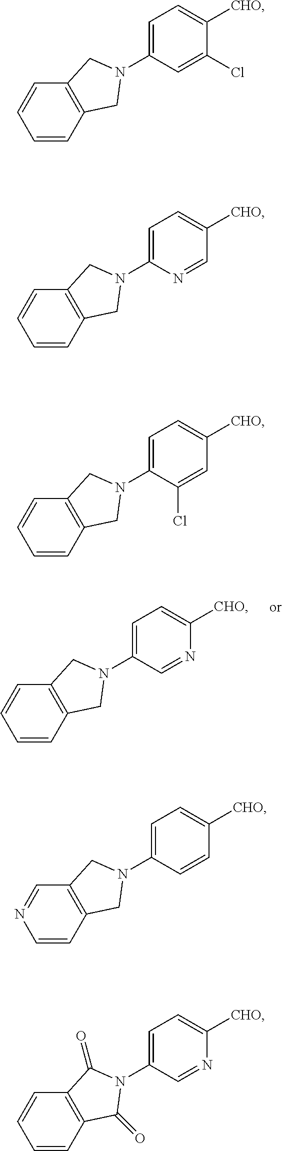 [SMILES: O=Cc1ccc(N2Cc3ccccc3C2)cc1Cl ; O=Cc1ccc(N2Cc3ccccc3C2)nc1 ; O=Cc1ccc(N2Cc3ccccc3C2)c(Cl)c1 ; O=Cc1ccc(N2Cc3ccccc3C2)cn1 ; O=Cc1ccc(N2Cc3ccncc3C2)cc1 ; O=Cc1ccc(N2C(=O)c3ccccc3C2=O)cn1]

Clause 16. The method of clause 1, wherein the ALDHi has a structure

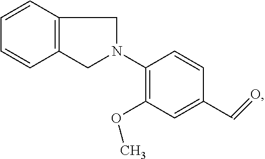 [SMILES: COc1cc(C=O)ccc1N1Cc2ccccc2C1]

or a pharmaceutically acceptable salt thereof.

Clause 17. An immunotherapy method for increasing an immune response to a pathogen or cancer cell antigen in a patient, comprising: administering to a patient an immunotherapeutic agent for increasing an antigen-specific immune response in a patient; and administering to the patient an adjuvant therapy to the administration of the immune cell, an amount of an aldehyde dehydrogenase-1A enzyme inhibitor (ALDHi) effective to reduce T_{REG} activity in the patient.

Clause 18. The method of clause 17, wherein the immunotherapeutic agent is an immune cell adapted to elicit an immune response to an antigen.

Clause 19. The method of clause 18, wherein the immunotherapeutic agent is an antigen-primed antigen-presenting cell (APC), such as a dendritic cell primed with an antigen.

Clause 20. The method of clause 18, wherein the immunotherapeutic agent is a CAR-T cell.

Clause 21. The method of clause 18, wherein the immunotherapeutic agent is a checkpoint inhibitor.

Clause 22. The method of clause 21, wherein the checkpoint inhibitor is a PD1- or PDL1-targeting antibody, such as pembrolizumab, nivolumab, atezolizurnab, or durvalumab.

Clause 23. The method of any one of clauses 17-22, wherein the antigen is an antigen of a cancer cell of the patient.

Clause 24. The method of any one of clauses 17-23, wherein the ALDHi has the structure:

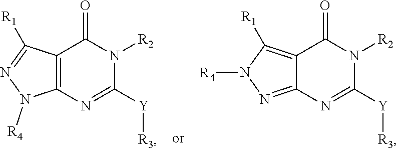

including pharmaceutically acceptable salts, solvates, and/or prodrugs thereof, wherein R₁, R₂, R₃, R₄, or Y independently can include any chemical moiety that permits the resulting compound to inhibit ALDH activity.

Clause 25. The method of clause 24, wherein R₁, R₂, R₃, R₄, or Y, independently, are any chemical moiety as described herein, and wherein Y may be present or absent.

Clause 26. The method of clause 24 or 25, wherein R₁ is H or C₁-C₃ alkyl (substituted or non-substituted).

Clause 27. The method of any one of clauses 24-26, wherein R₂ is H, C₀-C₃-alkyl-aryl or C₀-C₃-alkyl-heteroaryl, wherein the alkyls can be independently substituted or non-substituted, such as CH₃, or selected from one of the following

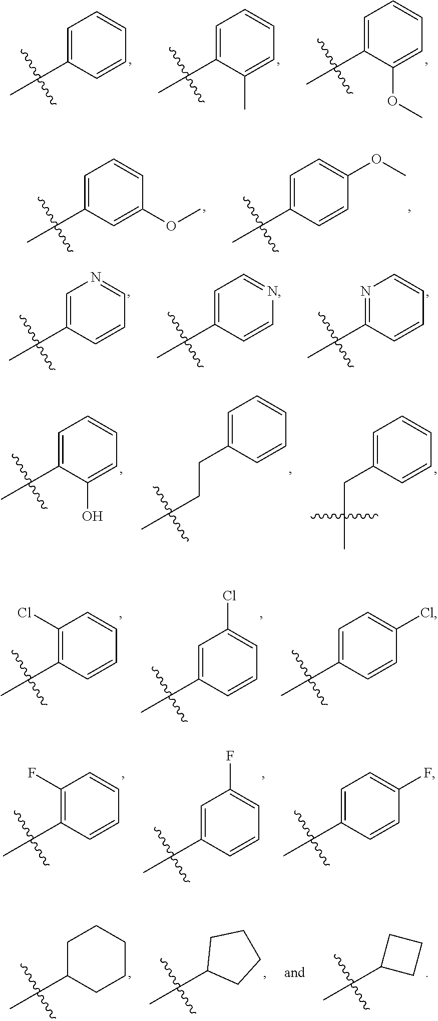

Clause 28. The method of any one of clauses 24-27, wherein Y is a linker, and optionally an alkyl, alkenyl, heteroalkyl, or heteroalkenyl, where the hetero atom is S, N, or O that is optionally substituted, such as a linker shown herein.

Clause 29. The method of any one of clauses 24-28, wherein R₃ is H, C₀-C₈ alkyl, C₀-C₂-alkyl-aryl or C₀-C₂-alkyl-heteroaryl, wherein the alkyls are independently substituted or non-substituted, such as a moiety as shown herein for R₃.

Clause 30. The method of any one of clauses 24-29, wherein R₄ is H, C₁-C₆ alkyl or cycloalkyl, optionally substituted, wherein one or more carbons may be replaced by oxygen, and wherein the alkyls are independently substituted or non-substituted, such as a structure of one of the following:

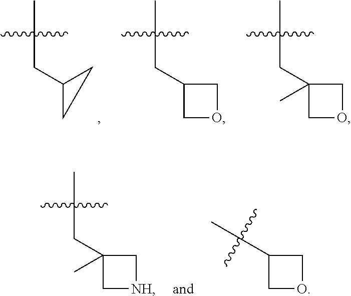

Clause 31. The method of any one of clauses 17-23, wherein the ALDHi is selected from one of the following, or a pharmaceutically acceptable salt thereof: 6-((3-fluoroberzyl)thio)-5-(o-tolyl)-1H-pyrazolo[3,4-d]pyrimidin-4(5H)-one, 6-((3-fluorobenzyl)thio)-5-phenyl-1H-pyrazolo[3,4-d]pyrimidin-4(5H)-one, 6-((3-fluorobenzyl)thio)-1-ethyl-5-(o-tolyl)-1H-pyrazolo[3,4-d]pyrimidin-4(5H)-one, 6-((3-fluorobenzyl)thio)-2-methyl-5-(o-tolyl)-2H-pyrazolo[3,4-d]pyrimidin-4(5H)-one, 1-ethyl-((3-fluorobenzyl)thio)-5-(0-tolyl)-1H-pyrazolo[3,4-d]pyrimidin-4(5H)-one, 2-ethyl-6-((3-fluorobenzyl)thio)-5-(o-tolyl)-2H-pyrazolo[3,4-d]pyrimidin-4(5H)-one, 1-(cyclopropylmethyl)-6-((3-fluorobenzyl)thio)-5-(o-tolyl)˜1H-pyrazolo[3,4-d]pyrimidin-4(5H)-one, 2-(cyclopropylmethyl)-6-((3-fluorobenzyl)thio)-5-(o-tolyl)-2H-pyrazolo[3,4-d]pyrimidin-4(5H)-one, 6-(3-fluorobenzyl)thio)-1-(oxetan-3-yl)-5-(o-tolyl)-1H-pyrazolo[3,4-d]pyrimidin-4(5H)-one, 6-((3-fluorobenzyl)thio)-2-(oxetan-3-yl)-5-(o-tolyl)-2H-pyrazolo[3,4-d]pyrimidin-4(5H)-one, 6-((3-fluorobenzyl)thio)-1-(oxetan-3-ylmethyl)-5-(o-tolyl-1H-pyrazolol[3,4-d]pyrimidin-4(5H)-one, 6-((3-fluorobenzyl)thio)-2-(oxetan-3-ylmethyl)-5-(o-tolyl)-2H-pyrazolo[3,4-d]pyrimidin-4(5H)-one, 6-((3-fluorobenzyl)thio)-2-methyl-5-phenyl-2H-pyrazolo[3,4-d]pyrimidin-4(5H)-one, 1-ethyl-6-((3-fluorobenzyl)thio)-5-phenyl-1H-pyrazolo[3,4-d]pyrimidin-4(5H)-one, 2-ethyl-6-((3-fluorobenzyl)thio)-5-phenyl-2H-pyrazolo[3,4-d]pyrimidin-4(5H)-one, 1-(cyclopropylmethyl)-6-((3-fluorobenzyl)thio)-5-phenyl-1H-pyrazolo[3,4-d]pyrimidin-4(5H)-one, 2-(cyclopropylmethyl)-6-((3-fluorobenzyl)thio)-5-phenyl-2H-pyrazolo[3,4-d]pyrimidin-4(5H)-one. 6-((3-fluorobenzyl)thio)-1-(oxetan-3-yl)-5-phenyl-1H-pyrazolo[3,4-d]pyrimidin-4(5H)-one, 6-((3-fluorobenzyl)thio)-2-(oxetan-3-yl)-5-phenyl-2H-pyrazolo[3,4-d]pyrimidin-4(5H)-one, 6-((3-fluorobenzyl)thio)-1-(oxetan-3-ylmethyl)-5-phenyl-1H-pyrazolo[3,4-d]pyrimidin-4(5H)-one, 6-((3-fluorobenzyl)thio)-2-(oxetan-3-ylmethyl)-5-phenyl-2H-pyrazolo[3,4-d]pyrimidin-4(5H)-one, 6-((3-fluorobenzyl)amino)-5-(o-tolyl)-1H-pyrazolo[3,4-d]pyrimidin-4(5H)-one, 6-((3-fluorophenethyl)amino)-1-methyl-5-(o-tolyl)-1H-pyrazolo[3,4-d]pyrimidin-4(5H)-one, 6-((3-fluorobenzyl)oxy)-1-methyl-5-(o-tolyl)-1H-pyrazolo[3,4-d]pyrimidin-4(5H)-one, 6-((3-fluorophenoxy)methyl)-1-methyl-5-phenyl-1H-pyrazolo[3,4-d]pyrimidin-4(5H)-one, 6-(((3-fluorobenzyl)amino)methyl)-methyl-5-phenyl-1H-pyrazolo[3,4-d]pyrimidin-4(5H)-one, 6-((3-fluorobenzyl)thio)-1-methyl-5-phenyl-1H-pyrazolo[3,4-d]pyrimidin-4(5H)-one, 6-((3-fluorobenzyl)thio)-5-(2-methoxyphenyl)-1-methyl-1H-pyrazolo[3,4-d]pyrimidin-4(5H)-one, 6-((3-fluorobenzyl)thio)-5-(3-methoxyphenyl)-1-methyl-1H-pyrazolo[3,4-d]pyrimidin-4(5H)-one, 6-((3-fluorobenzyl)thio)-5-(4-methoxyphenyl)-1-methyl-1H-pyrazolo[3,4-d]pyrimidin-4(5H)-one 6-((3-fluorobenzyl)thio)-methyl-5-(pyridin-3-yl)-1H-pyrazolo[3,4-d]pyrimidin-4(5H)-one, 6-((3-fluorobenzyl)thio)-1-methyl-5-(pyrimidin-4-yl)-1H-pyrazolo[3,4-d]pyrimidin-4(5H)-one, 6-((3-fluorobenzyl)thio)-5-(2-hydroxyphenyl)-1-methyl-1H-pyrazolo[3,4-d]pyrimidin-4(5H)-one, 6-((3-fluorobenzyl)thio)-1-methyl-5-(pyridin-2-yl)-1H-pyrazolo[3,4-d]pyrimidin-4(5H)-one, 6-((3-fluorobenzyl)thio)-1-methyl-5-phenethyl-1H-pyrazolo[3,4-d]pyrimidin-4(5H)-one, 5-benzyl-6-((3-fluorobenzyl)thio)-1-methyl-1H-pyrazolo[3,4-d]pyrimidin-4(5H)-one, 6-(3-fluorobenzyl)thio)-1,5-dimethyl-1H-pyrazolo[3,4-d]pyrimidin-4(5H)-one, 6-(isopentylthio)-1-methyl-5-phenyl-1H-pyrazolo[3,4-d]pyrimidin-4(5H)-one, 6-((3-fluorobenzyl)thio)-3-methyl-5-(o-tolyl)-2H-pyrazolo[3,4-d]pyrimidin-4(5H)-one, 6-(benzo[b]thiophen-2-ylmethoxy)-1-methyl-5-(o-tolyl)-1H-pyrazolo[3,4-d]pyrimidin-4(5H)-one, 5-(2-chlorophenyl)-6-(3-fluorobenzylthio)-1-methyl-1H-pyrazolo[3,4-d]pyrimidin-4(5H)-one, 5-(2-chlorophenyl)-6-((3-fluorobenzyl)thio)-1-methyl-1H-pyrazolo[3,4-d]pyrimidin-4(5H)-one, 5-(3-chlorophenyl)-6-((3-fluorobenzyl)thio)-1-methyl-1H-pyrazolo[3,4-d]pyrimidin-4(5H)-one, 5-(4-chlorophenyl)-6-((3-fluorobenzyl)thio)-1-methyl-1H-pyrazolo[3,4-d]pyrimidin-4(5H)-one, 6-((3-fluorobenzyl)thio)-5-(2-fluorophenyl)-1-methyl-1H-pyrazolo[3,4-d]pyrimidin-4(5H)-one, 5-cyclohexyl-6-((3-fluorobenzyl)thio)-1-methyl-1H-pyrazolo[3,4-d]pyrimidin-4(5H)-one, 5-cyclopentyl-6-((3-fluorobenzyl)thio)-1-methyl-1H-pyrazolo[3,4-d]pyrimidin-4(5H)-one, 5-cyclobutyl-6-((3-fluorobenzyl)thio)-1-methyl-1H-pyrazolo[3,4-d]pyrimidin-4(5H)-one, (E)-6-(3-fluorostyryl)-1-methyl-5-phenyl-1H-pyrazolo[3,4-d]pyrimidin-4(5H)-one, 6-(3-fluorophenethyl)-1-methyl-5-phenyl-1H-pyrazolo[3,4-d]pyrimidin-4(5H)-one, 6-(((3-fluorophenyl)amino)methyl)-1-methyl-5-phenyl-1H-pyrazolo[3,4-d]pyrimidin-4(5H)-one, 6-(((3-fluorobenzyl)oxy)methyl)-1-methyl-5-phenyl-1H-pyrazolo[3,4-d]pyrimidin-4(5H)-one, 6-(((3-fluorophenyl)thio)methyl)-1-methyl-5-phenyl-1H-pyrazolo[3,4-d]pyrimidin-4(5H)-one, 1-methyl-6-((2-oxo-2-phenylethyl)thio)-5-phenyl-1H-pyrazolo[3,4-d]pyrimidin-4(5H)-one, 3-(((1-methyl-4-oxo-5-phenyl-4,5-dihydro-1H-pyrazolo[3,4-d]pyrimidin-6-yl)thio)methyl)benzonitrile, 6-((3-methoxybenzyl)thio)-1-methyl-5-phenyl-1H-pyrazolo[3,4-d]pyrimidin-4(5H)-one, 6-(benzylthio)-1-methyl-5-phenyl-1H-pyrazolo[3,4-d]pyrimidin-4(5H)-one, 1-methyl-5-phenyl-6-((3-(2,2,2-trifluoroacetyl)benzyl)thio)-1H-pyrazolo[3,4-d]pyrimidin-4(5H)-one, 3-(((1-methyl-4-oxo-5-phenyl-4,5-dihydro-1H-pyrazolo[3,4-d]pyrimidin-6-yl)thio)methyl)benzaldehyde, 4-(((1-methyl-4-oxo-5-phenyl-4,5-dihydro-1H-pyrazolo[3,4-d]pyrimidin-6-yl)thio)methyl)benzaldehyde, 6-((3-fluorobenzyl)oxy)-1-methyl-5-phenyl-1H-pyrazolo[3,4-d]pyrimidin-4(5H)-one, 1-methyl-5-phenyl-6-((1-phenylethyl)thio)-1H-pyrazolo[3,4-d]pyrimidin-4(5H)-one, 6-((3-fluorophenyl)(methyl)amino)methyl)-1-methyl-5-phenyl-1H-pyrazolo[3,4-d]pyrimidin-4(5H)-one, 6-((3-fluorobenzyl)oxy)-2-(oxetan-3-ylmethyl)-5-phenyl-2H-pyrazolo[3,4-d]pyrimidin-4(5H)-one, 2-(oxetan-3-ylmethyl)-5-phenyl-6-((1-phenylethyl)thio)-2H-pyrazolo[3,4-d]pyrimidin-4(5H)-one, (R)-1-methyl-5-phenyl-6-((1-phenylethyl)thio)-1H-pyrazolo[3,4-d]pyrimidin-4(5H)-one, (S)-1-methyl-5-phenyl-6-((1-phenylethyl)thio)-1H-pyrazolo[3,4-d]pyrimidin-4(5H)-one, 1-methyl-5-phenyl-6-(1-phenylethoxy)-1H-pyrazolo[3,4-d]pyrimidin-4(5H)-one, 6-((3-fluorobenzyl)thio)-2-((3-methyloxetan-3-yl)methyl)-5-phenyl-2H-pyrazolo[3,4-d]pyrimidin-4(5H)-one, 3-((6-((3-fluorobenzyl)thio)-4-oxo-5-phenyl-4,5-dihydro-2H-pyrazolo[3,4-d]pyrimidin-2-yl)methyl)azetidin-1-ium sulfate, 1-methyl-5-phenyl-6-((1-phenylpropyl)thio)-1H-pyrazolo[3,4-d]pyrimidin-4(5H)-one, 6-((1-(3-fluorophenyl)ethyl)thio)-1-methyl-5-phenyl-1H-pyrazolo[3,4-d]pyrimidin-4(5H)-one, 6-(cyclohexylthio)-1-methyl-5-phenyl-1H-pyrazolo[3,4-d]pyrimidin-4(5H)-one, 1-(oxetan-3-yl)-5-phenyl-6-((1-phenylethyl)thio)-1H-pyrazolo[3,4-d]pyrimidin-4(5H)-one, 2-(oxetan-3-yl)-5-phenyl-6-((1-phenylethyl)thio)-2H-pyrazolo[3,4-d]pyrimidin-4(5H)-one, 1-methyl-6-((2-oxocyclopentyl)thio)-5-phenyl-1H-pyrazolo[3,4-d]pyrimidin-4(5H)-one, 1-methyl-6-((2-oxocyclohexyl)thio)-5-phenyl-1H-pyrazolo[3,4-d]pyrimidin-4(5H)-one, 1-methyl-5-phenyl-6-((2-phenylpropan-2-yl)thio)-1H-pyrazolo[3,4-d]pyrimidin-4(5H)-one, 6-((1-(3-hydroxyphenyl)ethyl)thio)-1-methyl-5-phenyl-1H-pyrazolo[3,4-d]pyrimidin-4(5H)-one, 1-methyl-5-phenyl-6-((1-(pyridin-2-yl)ethyl)thio)-1H-pyrazolo[3,4-d]pyrimidin-4(5H)-one, and 1-methyl-5-phenyl-6-((1-(pyridin-3-yl)ethyl)thio)-1H-pyrazolo[3,4-d]pyrimidin-4(5H)-one.

Clause 32. The method of any one of clauses 17-23, wherein the ALDHi is selected from a structure as depicted in FIGS. 2A-2C, or a pharmaceutically acceptable salt thereof:

Clause 33. The method of any one of clauses 17-23, wherein the ALDHi is selected from 673A, 257913 (913), 258085 (085), 262548 (548), 263052 (052), 263117 (117), 263118 (118), 263119 (119), 259122 (122), 263646 (646), 264202 (202), or a pharmaceutically acceptable salt thereof:

Clause 34. The method of any one of clauses 17-23, wherein the ALDHi is 673A, or a pharmaceutically acceptable salt thereof.

Clause 35. The method of any one of clauses 17-23, wherein the ALDHi has the structure:

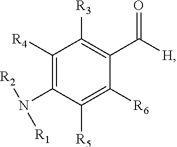

wherein R₁ and R₂ are independently or together, H, an alkyl, a cycloalkyl, together form a cycloalkyl or heterocyclic alkyl or aryl, an aryl, an alkenyl, a cycloalkyl, alkynl, or a substituted version of the aforementioned groups and R₃-R₆ are independently a halogen, H, an alkyl, a cycloalkyl, an aryl, an alkenyl, a cycloalkyl, alkynl, or a substituted version of the aforementioned groups, or a pharmaceutically acceptable salt thereof.

Clause 36. The method of clause 35, wherein the ALDHi has the structure:

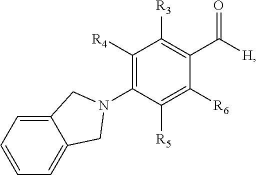

wherein and R₃-R₆ are independently a halogen, H, an alkyl, a cycloalkyl, an aryl, an alkenyl, a cycloalkyl, alkynl, or a substituted version of the aforementioned groups, or a pharmaceutically acceptable salt thereof.

Clause 37. The method of any one of clauses 17-23, wherein the ALDHi has a structure selected from:

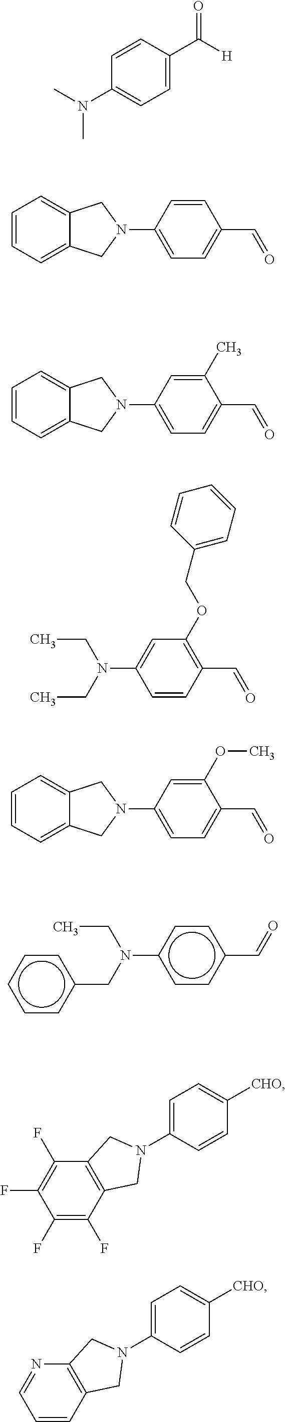

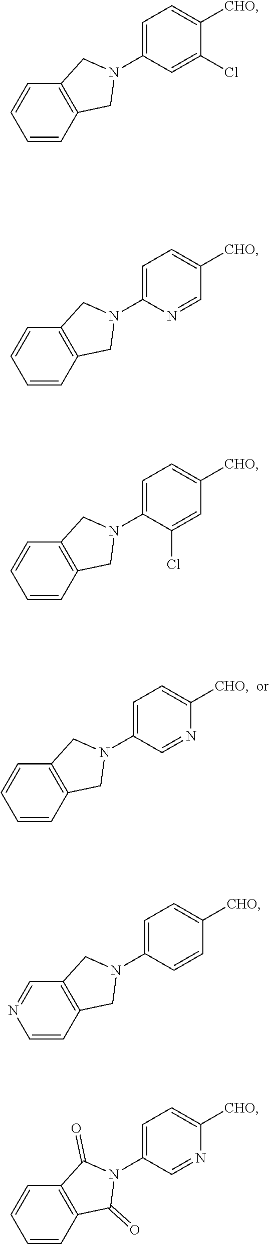

or a pharmaceutically acceptable salt thereof.

Clause 38. The method of any one of clauses 1-7, wherein the ALDHi has a structure:

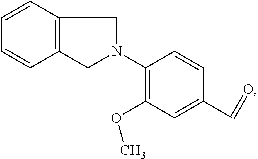 [SMILES: COc1cc(C=O)ccc1N1Cc2ccccc2C1]

or a pharmaceutically acceptable salt thereof.

Clause 39. The method of any one of clauses 17-38, for treating cancer in a patient, comprising: obtaining an antigen-presenting cell or a precursor thereof from the patient, wherein when an antigen-presenting cell precursor is obtained from the patient, further comprising culturing the precursor ex vivo in differentiation medium to differentiate the precursor to an antigen-presenting cell; culturing the antigen-presenting cell in the presence of an antigen of a cancer cell from the patient to produce a matured antigen-presenting cell; and administering the matured antigen-presenting cell to the patient with the ALDHi as an adjuvant therapy.

Clause 40. The method of clause 39, wherein the antigen-presenting cell or a precursor thereof is a dendritic cell or a precursor thereof.

Clause 41. The method of clause 39, wherein the antigen-presenting cell or a precursor thereof is a bone marrow progenitor cell, optionally obtained from the patient, and the method comprises differentiating the bone marrow progenitor cell to a dendritic cell.

Clause 42. The method of any one of clauses 17-41, wherein the immunotherapy is repeated one or more additional times.

Clause 43. The method of any one of clauses 17-42, wherein the ALDHi is administered to the patient on the same day as each instance of the immunotherapy (DO), and optionally for from one to ten days (D1-D10) after the immunotherapy.

Clause 44. The method of clause 43, wherein the ALDHi is administered from DO to D5, as multiple doses or a continuous dose.

Clause 45. The method of any one of clauses 17-44, wherein the patient has a tumor, and the ALDHi is administered in an amount effective to reduce tumor volume after 100 days in a patient, as compared to a tumor in a patient that is not treated with the ALDHi.

Clause 46. Use of an ALDHi as an adjuvant therapy to an immunotherapy to increase an immune response to a pathogen or cancer cell antigen in a patient, optionally according to a method of any one of clauses 1-45.

Clause 47. An ALDHi for use in an adjuvant therapy to an immunotherapy.

### BRIEF DESCRIPTION OF THE DRAWINGS

FIGS. 1A-1C provide structures for Group R₃, as described below.

FIGS. 2A-2C provide exemplary ALDHi structures.

FIGS. 3A-3C: ALDHi promotes inflammatory cell death in tumor cells. FIG. 3A Heat map (log₂ fold change vs. control) of significantly enriched genes differentially expressed in ALDHi treated cells. FIG. 3B (i) IPA analysis identifying top regulators/pathways and functions modified by ALDHi, FIG. 3B (ii) Heat map (log 2 fold change vs. control) of selected genes significantly differentially expressed in ALDHi treated cells. FIG. 3C ALDHi can increase the expression of ATF3, ATF4, Phospho-eIF2α.

FIGS. 4A-4C and 5A and 5B: Effect of ALDHi treatment on anti-CD3/CD28 stimulated splenocytes. FIGS. 4A-4C Two independent experiments showing the efficacy of ALDHi on anti-CD3/CD28 stimulated splenocytes for proliferation of CD4+ and CD8+ cells determined by flow cytometry. ALDHi increase CD8 and decrease CD4, this is partially abrogated by retinoic acid (RA) suggesting an on target effect. FIGS. 5A and 5B. Spleen-isolated naïve CD4+ T cells were subjected to Treg inducible conditions (IL2, TGFβ) with selected ALDHi from FIG. 4A-4C Percent Foxp3 positive Treg cells are shown as dot plots and their normalized percent is shown in table.

FIG. 6 Efficacy of ALDHi on anti-CD3/CD28 stimulated splenocytes for proliferation of CD4+ and CD8+ cells determined by flow cytometry.

FIG. 7 DC preventive vaccine for ID8 sub-Q tumor model. A Experimental design of bone-marrow DCs cell generation and maturation for DC vaccine. B Therapeutic schema in vivo for 3 different groups.

FIG. 8 ALDHi as adjuvant to tumor vaccine increases tumor growth control.

FIG. 9 provides an outline of the ascites experiment as described below.

FIGS. 10A-10F ALDHi effect on ascites. FIG. 10A Gating strategy for flow data analysis. FIG. 10B-10E 673A dramatically reduced population of immune suppressive CD14 cells. FIG. 10F Western blot analysis shows the reduction of the immunosuppressive master regulator NR4A1 level upon ALDHi treatment in ascites samples.

FIG. 11 ALDHi effect on bone-marrow dendritic cells (BMDC). A graph showing BMDC proliferation upon ALDHi treatment is depicted.

FIG. 12 ALDHi effect on bone-marrow DCs. A graph showing BMDC proliferation upon ALDHi treatment is provided.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. As used herein “a” and “an” refer to one or more.

As used herein, the terms “comprising,” “comprise” or “comprised,” and variations thereof, are open ended and do not exclude the presence of other elements not identified. In contrast, the term “consisting of” and variations thereof is intended to be closed, and excludes additional elements in anything but trace amounts.

As used herein, the term “patient” or “subject” refers to members of the animal kingdom including but not limited to human beings and “mammal” refers to all mammals, including, but not limited to human beings.

As used herein, the “treatment” or “treating”, means administration to a patient by any suitable dosage regimen, procedure and/or administration route of a composition, device or structure with the object of achieving a desirable clinical/medical end-point, including but not limited to, reduction of symptoms or normalization (moving towards a normal range) of one or more clinical values. In the context of treatment of cancer, treatment includes reducing tumor size or number, or reducing overall cancer cell counts.

Drug products, or pharmaceutical compositions comprising an active agent (e.g., drug), for example, an active agent, such as an ALDHi as described herein, that decreases immune suppressive, CD4+ regulatory T cell (T_{REG}) numbers or activity in a patient, may be prepared by any method known in the art of pharmacy, for example, by bringing into association the active ingredient with the carrier(s) or excipient(s). As used herein, a “pharmaceutically acceptable excipient”, “carrier” or “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable excipients include one or more of water, saline, phosphate buffered saline, dextrose, glycerol, sugars, polyalcohols such as mannitol, sorbitol, sodium chloride, or ethanol, as well as combinations thereof. Pharmaceutically acceptable carriers may further comprise minor amounts of auxiliary substances such as wetting or emulsifying agents, preservatives or buffers, which enhance the shelf life or effectiveness of the active agent. An active compound may be prepared with a carrier that will protect the compound against rapid release, such as a controlled release formulation, including implants, transdermal patches, and microencapsulated delivery systems. Biodegradable, biocompatible polymers can be used in delivery systems, such as, for example, ethylene vinyl acetate, polyanhydrides, polyglycolic acid, collagen, polyorthoesters, and polylactic acid. Methods for the preparation of such formulations are broadly-known to those skilled in the art.

Additionally, active agent-containing compositions may be in variety of forms. A preferred form depends on the intended mode of administration and therapeutic application, which will in turn dictate the choices of carriers/excipients. Suitable forms include, but are not limited to, liquid, semi-solid and solid dosage forms.

Pharmaceutical formulations adapted for parenteral administration include aqueous and non-aqueous sterile injection solutions which may contain, for example and without limitation, anti-oxidants, buffers, bacteriostats, lipids, liposomes, emulsifiers, suspending agents, and rheology modifiers. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets.

Described herein are immunotherapeutic methods useful in the treatment of diseases, such as cancer. As a class, immunotherapies include immunotherapies for treatment of cancer uses immune cells to target cancer cells. The methods described herein are used to enhance tumor targeting immune responses.

To increase an immune response, or to reduce or eliminate that immunosuppressive environment, adjuvant therapy of a patient with an ALDHi may precede, coincide, or follow administration of immunotherapy reagents to the patient in order to increase the patient's cancer-specific immune response and/or to suppress detrimental T_{REG} activity. By administering an ALDHi as an adjuvant to an immunotherapy, a more robust immune response directed to a cancer antigen is expected, at least in part by decreasing T_{REG} cell populations, and increasing populations of cytotoxic CD8 T cells, DC and/or subsets of CD4 helper (T_{H}) T cells.

In one example, effective cancer vaccines require the development of a robust antigen-specific immune response, for which down-regulation of T_{Reg} activity, activation of CD8 cells, and/or T_{H} cells and induction of DC is desired. By administering an ALDHi as an adjuvant therapy to an immunotherapy, a more robust immune response directed to a cancer antigen is expected, at least in part by decreasing T_{REG} cell populations, and increasing populations of CD8 T cells, DC, and/or T_{H} cells. The ALDHi may be administered according to any effective dosage regimen, such as a bolus, prior to, after, or during administration of an immunotherapy reagent, continuously at any time before, during, or after administration of the immunotherapy reagent, or intermittently according to any effective dosing regimen before, during, or after administration of the immunotherapy reagent.

As indicated below, adjuvant therapy with an ALDHi in combination with ex vivo-generated DCs loaded with tumor antigen significantly reduces tumor growth in the described tumor model. DC immunotherapies that rely on antigen recognition, and which rely on the generation of an immune response to an antigen, are expected to benefit from use of an ALDHi adjuvant therapy. DC-based vaccines in humans can be restricted by the ability to harvest sufficient numbers of DCs. The ability to use ALDHi treatment of DC ex-vivo to expand DC numbers can offer significant benefit. In one example, the DC immunotherapy involves administration of ex vivo-generated DCs loaded with tumor antigens.

As above, a limitation of CAR-T therapies is the immunosuppressive microenvironment associated with malignancies. To reduce or eliminate that immunosuppressive environment, adjuvant therapy of a patient with an ALDHi may precede, coincide, or follow administration of CAR-T cells to the patient in order to increase the patient's cancer-specific immune response and/or to suppress T_{REG} activity that suppresses recognition of tumor antigens by the patient's immune system, and which may inhibit CAR-T cell activity. By administering an ALDHi as an adjuvant to CAR-T therapy, a more robust immune response directed to a cancer antigen is expected, at least in part by decreasing T_{REG} cell populations, and promoting proliferation of cytotoxic CD8 T cells, DC, and/or T_{H} cells, including the therapeutic, ex vivo modified CAR-modified T-cells.

As above, another form of cancer treatment is the use of T cell-targeted immunomodulators that block immune checkpoints like CTLA-4, PD1 or PDL1. Therapies that increases the patient's immune response, used in conjunction with checkpoint inhibitor therapeutics, would be beneficial. IN the context of the present disclosure, administration of an ALDHi concurrently with, e.g., before, after, or during administration of a checkpoint inhibitor would be expected to further enhance the immune response generated by the checkpoint inhibitor, alone. The ALDHi may be administered according to any effective dosage regimen, such as a bolus, prior to, after, or during administration of the checkpoint inhibitor, continuously at any time before, during, or after administration of the checkpoint inhibitor, or intermittently according to any effective dosing regimen before, during, or after administration of the checkpoint inhibitor.

Other immunotherapies, such as bi-specific T-cell engagers (BiTEs), that rely on elicitation of an immune response may be amenable to adjuvant therapy with ALDHis as described herein.

The overall number of an immune cell type can be proportional to activity of that cell type. As such the up-regulation or down-regulation of activity of a specific cell type or population may correspond to, or may be proportional to, a respective increase or decrease of the overall number of those cells, or a proportion of those cells relative to another cell population or class of cells, such as the overall number of T_{Reg} cells, T_{C} cells, or T_{H} cells relative to each other or to overall lymphocyte counts.

The ALDHis described herein are also shown in the examples below to be effective in expanding bone-marrow cells for production of DCs ex vivo. The ALDHi is included in an effective amount, such as >2 μM, e.g., from 5-20 μM, optionally in the presence of GM-CSF (granulocyte-macrophage colony-stimulating factor), to increase expansion of bone marrow cells, e.g., Dendritic Cells (DCs), as compared to equivalent culture in the absence of the ALDHi.

Inhibitors of aldehyde dehydrogenase-1A enzymes (ALDHis) include, without limitation, those compounds described in United States Patent Publication No. 2019/0255055, and in International Patent Publication No. WO 2017/223086 which are incorporated herein by reference for in their entirety for their technical disclosure (see, also Huddle B C, et al. Structure-Based Optimization of a Novel Class of Aldehyde Dehydrogenase 1A (ALDH1A) Subfamily-Selective Inhibitors as Potential Adjuncts to Ovarian Cancer Chemotherapy. J Med Chem. 2018 Oct. 11:61(19):8754-8773 and Huddle B C, et al. Development of 2,5-dihydro-4H-pyrazolo[3,4-d]pyrimidin-4-one inhibitors of aldehyde dehydrogenase 1A (ALDH1A) as potential adjuncts to ovarian cancer chemotherapy. Eur. J Med Chem. 2021 Feb. 5; 211:113060). Thiopyrimidinone compounds of Formulas I and II (See, United States Patent Publication No. 2019/0255055 and International Patent Publication No. WO 2017/223086) are non-limiting examples of ALDHis:

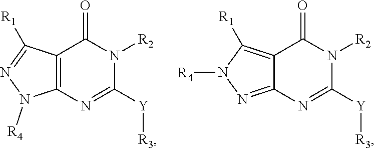

including pharmaceutically acceptable salts, solvates, and/or prodrugs thereof.

The particular chemical moiety for R₁, R₂, R₃, R₄, or Y independently can include any chemical moiety that permits the resulting compound to inhibit ALDH activity (e.g., one or more of ALDH1A1, ALDH1A2, ALDH1A3, ALDH2 activity). The particular chemical moiety for R₁, R₂, R₃, R₄, or Y independently may include any chemical moiety that permits the resulting compound to induce promote T-cell differentiation favoring cytotoxic CD8⁺ T cells (T_{C}), dendritic cells, and CD4⁺ T_{Helper} (T_{H}) cells, while disfavoring CD4⁺ T_{Regulatory} (T_{Reg}) cells.

R₁ can be H or C₁-C₃ alkyl (substituted or non-substituted). R₁ can be H, CH₃, CH₂CH₃, or CH₂CH₂CH₃.

R₂ can be H, C₀-C₃-alkyl-aryl, or C₀-C₃-alkyl-heteroaryl, wherein the alkyls can be independently substituted or non-substituted. R₂ can be phenyl (substituted or non-substituted). R₂ can be CH₃, or selected from one of the following:

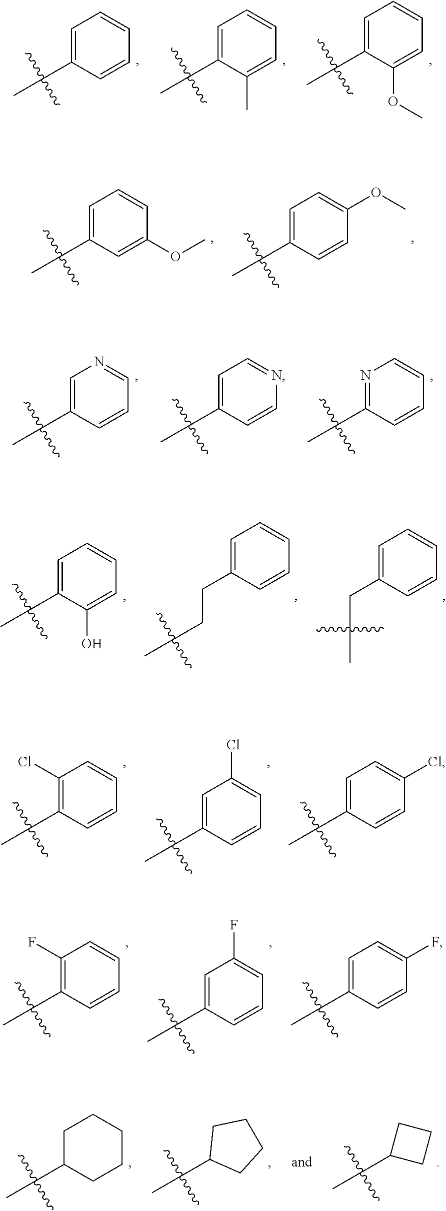

Y can be a linker that is present or absent. When Y is present, Y can be selected from:

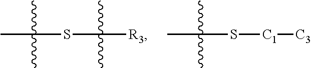

alkyl or alkenyl (substituted or unsubstituted) (branched or unbranched)

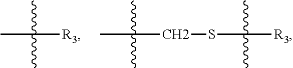

alkyl or alkenyl (substituted or unsubstituted) (branched or unbranched)

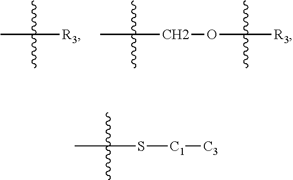

alkyl or alkenyl (substituted or unsubstituted) (branched or unbranched)

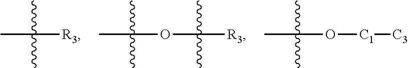

alkyl or alkenyl (substituted or unsubstituted) (branched or unbranched

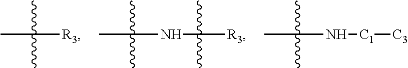

alkyl or alkenyl (substituted or unsubstituted) (branched or unbranched)

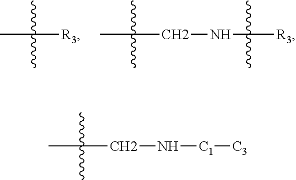

alkyl or alkenyl (substituted or unsubstituted) (branched or unbranched)

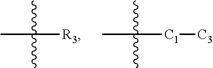

alkyl or alkenyl (substituted or unsubstituted) (branched or unbranched)

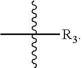

When Y is present, Y can be selected from

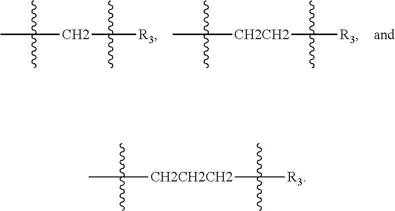

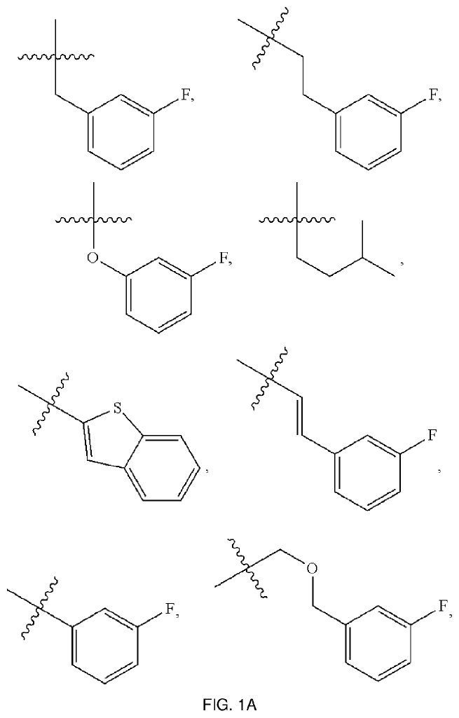

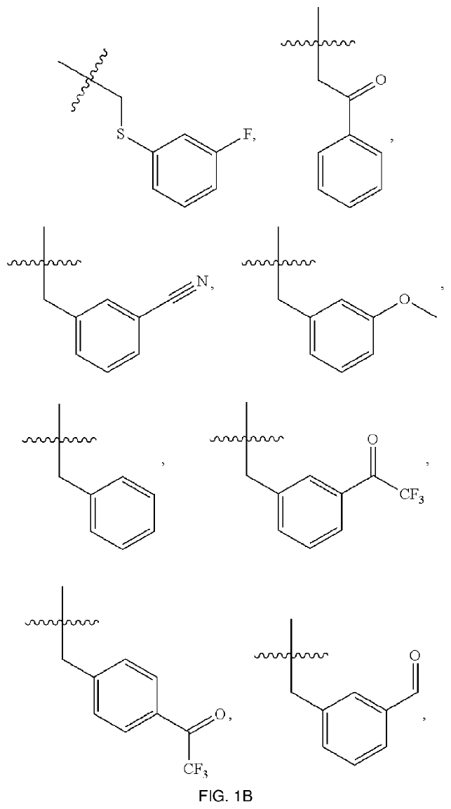

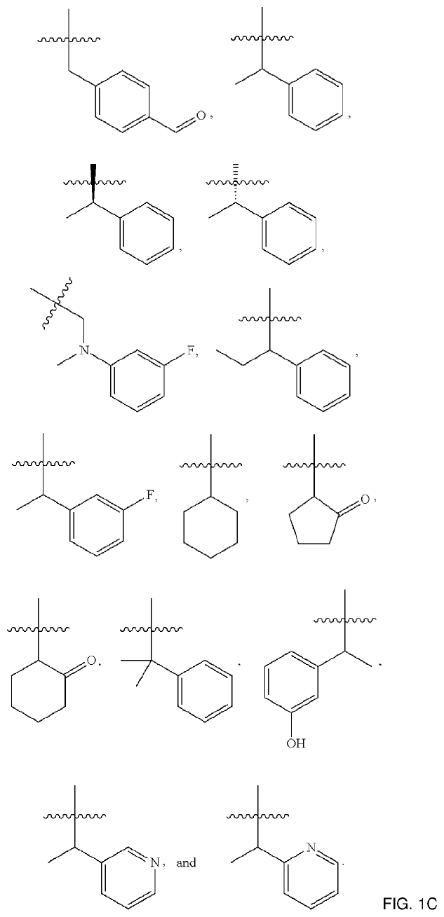

R₃ can be H, C₁-C₈ alkyl, C₀-C₂-alkyl-aryl or C₀-C₂-alkyl-heteroaryl, wherein the alkyls are independently substituted or non-substituted. R₃ can be C₁-alkyl-aryl or C₁-alkyl-heteroaryl. R₃ can be CH₃ or can have a structure chosen from a group as shown in FIGS. 1A-1C.

R₄ can be H, C₁-C₆ alkyl, or cycloalkyl, optionally substituted and wherein one or more carbons may be replaced by oxygen, wherein the alkyls are independently substituted or non-substituted. R₄ can be C₁-C₄ alkyl or cycloalkyl, wherein one or more carbons may be replaced by O. R₄ can be H, CH₃, or CH₂CH₃, or a structure selected from:

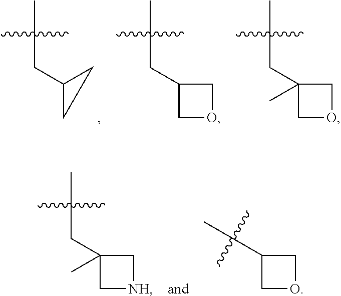

The thiopyrimidinone compounds can have the structure of Formulas I and II:

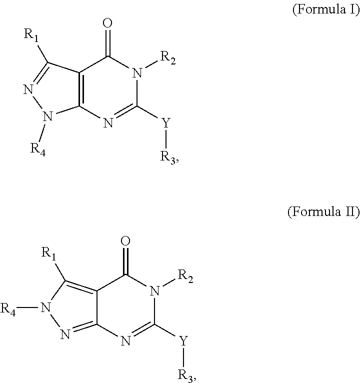

including pharmaceutically acceptable salts, solvates, and/or prodrugs thereof; wherein R₁, R₂, R₃, and Y are defined as above; wherein R₄ is H, C₁-C₆ alkyl, or cycloalkyl (optionally substituted or unsubstituted) (wherein one or more carbons may be replaced by oxygen or nitrogen). R₄ can be selected from H, CH₃, or CH₂CH₃, or have a structure selected from the following structures:

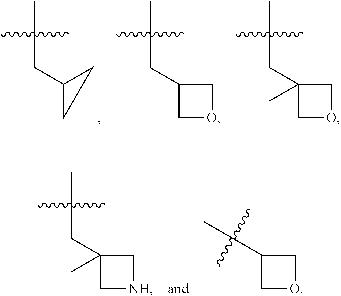

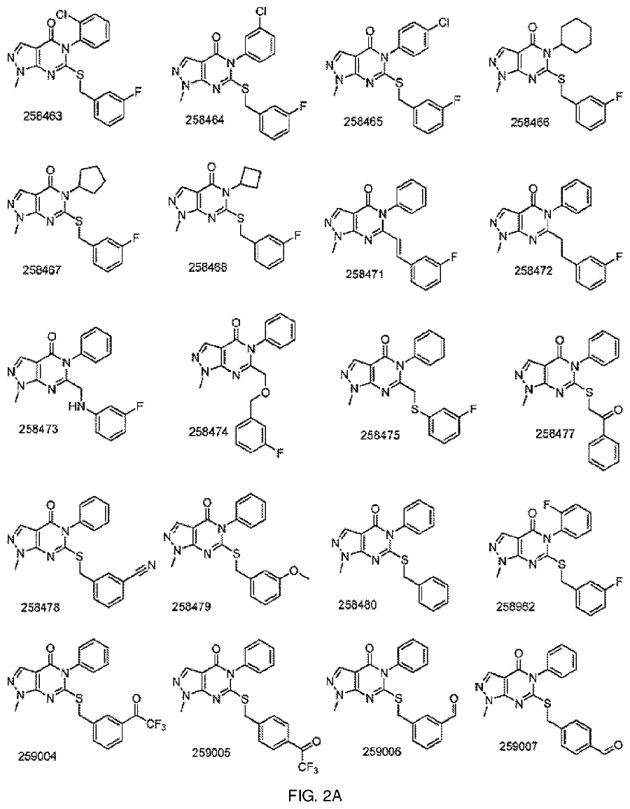

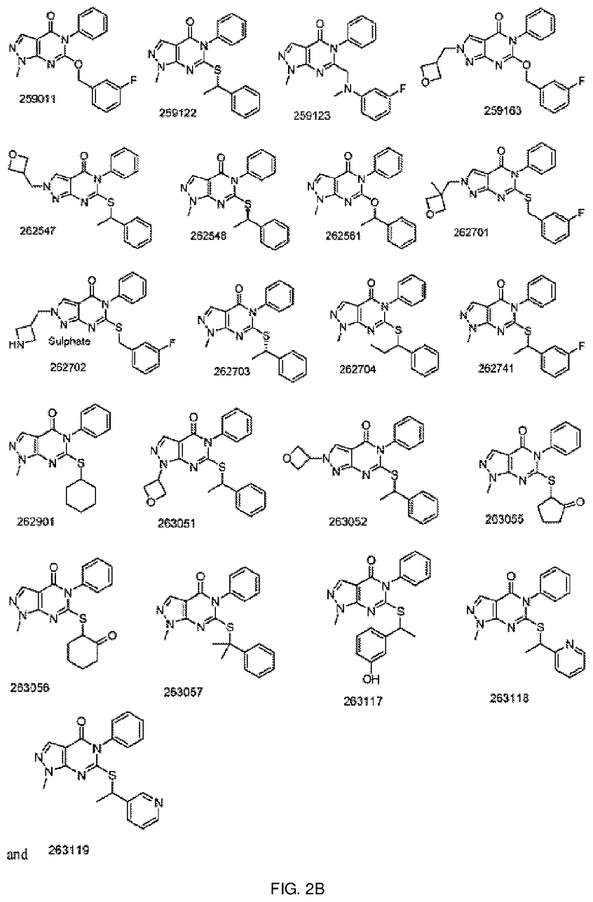

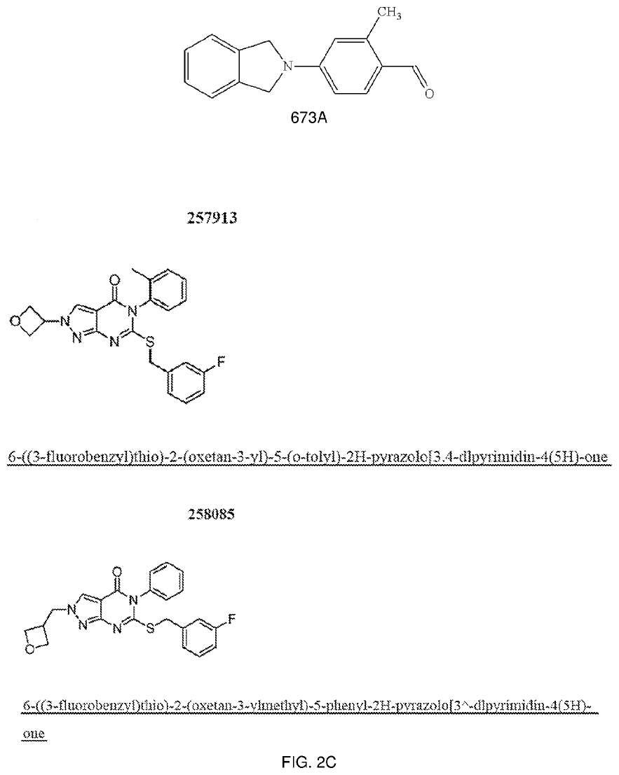

Non-limiting examples of ALDHis are depicted in FIGS. 2A-2C. Non-limiting examples of ALDHis also include: 6-((3-fluorobenzyl)thio)-5-(o-tolyl)-1H-pyrazolo[3,4-d]pyrimidin-4(5H)-one, 6-((3-fluorobenzyl)thio)-5-phenyl-1H-pyrazolo[3,4-d]pyrimidin-4(5H)-one, 6-((3-fluorobenzyl)thio)-1-methyl-5-(o-tolyl)-1H-pyrazolo[3,4-d]pyrimidin-4(5H)-one, 6-((3-fluorobenzyl)thio)-2-methyl-5-(o-tolyl)-2H-pyrazolo[3,4-d]pyrimidin-4(5H)-one, 1-ethyl-6-((3-fluorobenzyl)thio)-5-(o-tolyl)-1H-pyrazolo[3,4-d]pyrimidin-4(5H)-one, 2-ethyl-6-((3-fluorobenzyl)thio)-5-(o-tolyl)-2H-pyrazolo[3,4-d]pyrimidin-4(5H)-one, 1-(cyclopropylmethyl)-6-((3-fluorobenzyl)thio)-5-(o-tolyl)-1H-pyrazolo[3,4-d]pyrimidin-4(5H)-one, 2-(cyclopropylmethyl)-6-((3-fluorobenzyl)thio)-5-(o-tolyl)-2H-pyrazolo[3,4-d]pyrimidin-4(5H)-one, 6-((3-fluorobenzyl)thio)-1-(oxetan-3-yl)-5-(o-tolyl)-1H-pyrazolo[3,4-d]pyrimidin-4(5H)-one, 6-((3-fluorobenzyl)thio)-2-(oxetan-3-yl)-5-(o-tolyl)-2H-pyrazolo[3,4-d]pyrimidin-4(5H)-one, 6-((3-fluorobenzyl)thio)-1-(oxetan-3-ylmethyl)-5-(o-tolyl)-1H-pyrazolol[3,4-d]pyrimidin-4(5H)-one, 6-((3-fluorobenzyl)thio)-2-(oxetan-3-ylmethyl)-5-(o-tolyl)-2H-pyrazolo[3,4-d]pyrimidin-4(5H)-one, 6-((3-fluorobenzyl)thio)-2-methyl-5-phenyl-2H-pyrazolo[3,4-d]pyrimidin-4(5H)-one, 1-ethyl-6-((3-fluorobenzyl)thio)-5-phenyl-1H-pyrazolo[3,4-d]pyrimidin-4(5H)-one, 2-ethyl-6-((3-fluorobenzyl)thio)-5-phenyl-2H-pyrazolo[3,4-d]pyrimidin-4(5H)-one, 1-(cyclopropylmethyl)-6-((3-fluorobenzyl)thio)-5-phenyl-1H-pyrazolo[3,4-d]pyrimidin-4(5H)-one, 2-(cyclopropylmethyl)-6-((3-fluorobenzyl)thio)-5-phenyl-2H-pyrazolo[3,4-d]pyrimidin-4(5H)-one, 6-((3-fluorobenzyl)thio)-1-(oxetan-3-yl)-5-phenyl-1H-pyrazolo[3,4-d]pyrimidin-4(5H)-one, 6-((3-fluorobenzyl)thio)-2-(oxetan-3-yl)-5-phenyl-2H-pyrazolo[3,4-d]pyrimidin-4(5H)-one, 6-((3-fluorobenzyl)thio)-1-(oxetan-3-ylmethyl)-5-phenyl-1H-pyrazolo[3,4-d]pyrimidin-4(5H)-one, 6-((3-fluorobenzyl)thio)-2-(oxetan-3-ylmethyl)-5-phenyl-2H-pyrazolo[3,4-d]pyrimidin-4(5H)-one, 6-((3-fluorobenzyl)amino)-5-(o-tolyl)-1H-pyrazolo[3,4-d]pyrimidin-4(5H)-one, 6-((3-fluorophenethyl)amino)-1-methyl-5-(o-tolyl)-1H-pyrazolo[3,4-d]pyrimidin-4(5H)-one, 6-((3-fluorobenzyl)oxy)-1-methyl-5-(o-tolyl)-1H-pyrazolo[3,4-d]pyrimidin-4(5H)-one, 6-((3-fluorophenoxy)methyl)-1-methyl-5-phenyl-1H-pyrazolo[3,4-d]pyrimidin-4(5H)-one, 6-(((3-fluorobenzyl)amino)methyl)-1-methyl-5-phenyl-1H-pyrazolo[3,4-d]pyrimidin-4(5H)-one, 6-((3-fluorobenzyl)thio)-1-methyl-5-phenyl-1H-pyrazolo[3,4-d]pyrimidin-4(5H)-one, 6-((3-fluorobenzyl)thio)-5-(2-methoxyphenyl)-1-methyl-1H-pyrazolo[3,4-d]pyrimidin-4(5H)-one, 6-((3-fluorobenzyl)thio)-5-(3-methoxyphenyl)-1-methyl-1H-pyrazolo[3,4-d]pyrimidin-4(5H)-one, 6-((3-fluorobenzyl)thio)-5-(4-methoxyphenyl)-1-methyl-1H-pyrazolo[3,4-d]pyrimidin-4(5H)-one 6-((3-fluorobenzyl)thio)-1-methyl-5-(pyridin-3-yl)-1H-pyrazolo[3,4-d]pyrimidin-4(5H)-one, 6-((3-fluorobenzyl)thio)-1-methyl-5-(pyridin-4-yl)-1H-pyrazolo[3,4-d]pyrimidin-4(5H)-one, 6-((3-fluorobenzyl)thio)-5-(2-hydroxyphenyl)-1-methyl-1H-pyrazolo[3,4-d]pyrimidin-4(5H)-one, 6-((3-fluorobenzyl)thio)-1-methyl-5-(pyridin-2-yl)-1H-pyrazolo[3,4-d]pyrimidin-4(5H)-one, 6-((3-fluorobenzyl)thio)-1-methyl-5-phenethyl-1H-pyrazolo[3,4-d]pyrimidin-4(5H)-one, 5-benzyl-6-((3-fluorobenzyl)thio)-1-methyl-1H-pyrazolo[3,4-d]pyrimidin-4(5H)-one, 6-((3-fluorobenzyl)thio)-1,5-dimethyl-1H-pyrazolo[3,4-d]pyrimidin-4(5H)-one, 6-(isopentylthio)-1-methyl-5-phenyl-1H-pyrazolo[3,4-d]pyrimidin-4(5H)-one, 6-((3-fluorobenzyl)thio)-3-methyl-5-(o-tolyl)-2H-pyrazolo[3,4-d]pyrimidin-4(5H)-one, 6-(benzo[b]thiophen-2-ylmethoxy)-1-methyl-5-(o-tolyl)-1H-pyrazolo[3,4-d]pyrimidin-4(5H)-one, 5-(2-chlorophenyl)-6-(3-fluorobenzylthio)-1-methyl-1H-pyrazolo[3,4-d]pyrimidin-4(5H)-one, 5-(2-chlorophenyl)-6-((3-fluorobenzyl)thio)-1-methyl-1H-pyrazolo[3,4-d]pyrimidin-4(5H)-one, 5-(3-chlorophenyl)-6-((3-fluorobenzyl)thio)-1-methyl-1H-pyrazolo[3,4-d]pyrimidin-4(5H)-one, 5-(4-chlorophenyl)-6-((3-fluorobenzyl)thio)-1-methyl-1H-pyrazolo[3,4-d]pyrimidin-4(5H)-one, 6-((3-fluorobenzyl)thio)-5-(2-fluorophenyl)-1-methyl-1H-pyrazolo[3,4-d]pyrimidin-4(5H)-one, 5-cyclohexyl-6-((3-fluorobenzyl)thio)-1-methyl-1H-pyrazolo[3,4-d]pyrimidin-4(5H)-one, 5-cyclopentyl-6-((3-fluorobenzyl)thio)-1-methyl-1H-pyrazolo[3,4-d]pyrimidin-4(5H)-one, 5-cyclobutyl-6-((3-fluorobenzyl)thio)-1-methyl-1H-pyrazolo[3,4-d]pyrimidin-4(5H)-one, (E)-6-(3-fluorostyryl)-1-methyl-5-phenyl-1H-pyrazolo[3,4-d]pyrimidin-4(5H)-one, 6-(3-fluorophenethyl)-1-methyl-5-phenyl-1H-pyrazolo[3,4-d]pyrimidin-4(5H)-one, 6-(((3-fluorophenyl)amino)methyl)-1-methyl-5-phenyl-1H-pyrazolo[3,4-d]pyrimidin-4(5H)-one, 6-(((3-fluorobenzyl)oxy)methyl)-1-methyl-5-phenyl-1H-pyrazolo[3,4-d]pyrimidin-4(5H)-one, 6-(((3-fluorophenyl)thio)methyl)-1-methyl-5-phenyl-1H-pyrazolo[3,4-d]pyrimidin-4(5H)-one, 1-methyl-6-((2-oxo-2-phenylethyl)thio)-5-phenyl-1H-pyrazolo[3,4-d]pyrimidin-4(5H)-one, 3-(((1-methyl-4-oxo-5-phenyl-4,5-dihydro-1H-pyrazolo[3,4-d]pyrimidin-6-yl)thio)methyl)benzonitrile, 6-((3-methoxybenzyl)thio)-1-methyl-5-phenyl-1H-pyrazolo[3,4-d]pyrimidin-4(5H)-one, 6-(benzylthio)-1-methyl-5-phenyl-1H-pyrazolo[3,4-d]pyrimidin-4(5H)-one, 1-methyl-5-phenyl-6-((3-(2,2,2-trifluoroacetyl)benzyl)thio)-1H-pyrazolo[3,4-d]pyrimidin-4(5H)-one, 3-(((1-methyl-4-oxo-5-phenyl-4,5-dihydro-1H-pyrazolo[3,4-d]pyrimidin-6-yl)thio)methyl)benzaldehyde, 4-(((1-methyl-4-oxo-5-phenyl-4,5-dihydro-1H-pyrazolo[3,4-d]pyrimidin-6-yl)thio)methyl)benzaldehyde, 6-((3-fluorobenzyl)oxy)-1-methyl-5-phenyl-1H-pyrazolo[3,4-d]pyrimidin-4(5H)-one, 1-methyl-5-phenyl-6-((1-phenylethyl)thio)-1H-pyrazolo[3,4-d]pyrimidin-4(5H)-one, 6-(((3-fluorophenyl)(methyl)amino)methyl)-1-methyl-5-phenyl-1H-pyrazolo[3,4-d]pyrimidin-4(5H)-one, 6-((3-fluorobenzyl)oxy)-2-(oxetan-3-ylmethyl)-5-phenyl-2H-pyrazolo[3,4-d]pyrimidin-4(5H)-one, 2-(oxetan-3-ylmethyl)-5-phenyl-6-((1-phenylethyl)thio)-2H-pyrazolo[3,4-d]pyrimidin-4(5H)-one, (R)-1-methyl-5-phenyl-6-((1-phenylethyl)thio)-1H-pyrazolo[3,4-d]pyrimidin-4(5H)-one, (S)-1-methyl-5-phenyl-6-((1-phenylethyl)thio)-1H-pyrazolo[3,4-d]pyrimidin-4(5H)-one, 1-methyl-5-phenyl-6-(1-phenylethoxy)-1H-pyrazolo[3,4-d]pyrimidin-4(5H)-one, 6-((3-fluorobenzyl)thio)-2-((3-methyloxetan-3-yl)methyl)-5-phenyl-2H-pyrazolo[3,4-d]pyrimidin-4(5H)-one, 3-((6-((3-fluorobenzyl)thio)-4-oxo-5-phenyl-4,5-dihydro-2H-pyrazolo[3,4-d]pyrimidin-2-yl)methyl)azetidin-1-ium sulfate, 1-methyl-5-phenyl-6-((1-phenylpropyl)thio)-1H-pyrazolo[3,4-d]pyrimidin-4(5H)-one, 6-((1-(3-fluorophenyl)ethyl)thio)-1-methyl-5-phenyl-1H-pyrazolo[3,4-d]pyrimidin-4(5H)-one, 6-(cyclohexylthio)-1-methyl-5-phenyl-1H-pyrazolo[3,4-d]pyrimidin-4(5H)-one, 1-(oxetan-3-yl)-5-phenyl-6-((1-phenylethyl)thio)-1H-pyrazolo[3,4-d]pyrimidin-4(5H)-one, 2-(oxetan-3-yl)-5-phenyl-6-((1-phenylethyl)thio)-2H-pyrazolo[3,4-d]pyrimidin-4(5H)-one, 1-methyl-6-((2-oxocyclopentyl)thio)-5-phenyl-1H-pyrazolo[3,4-d]pyrimidin-4(5H)-one, 1-methyl-6-((2-oxocyclohexyl)thio)-5-phenyl-1H-pyrazolo[3,4-d]pyrimidin-4(5H)-one, 1-methyl-5-phenyl-6-((2-phenylpropan-2-yl)thio)-1H-pyrazolo[3,4-d]pyrimidin-4(5H)-one, 6-((1-(3-hydroxyphenyl)ethyl)thio)-1-methyl-5-phenyl-1H-pyrazolo[3,4-d]pyrimidin-4(5H)-one, 1-methyl-5-phenyl-6-((1-(pyridin-2-yl)ethyl)thio)-1H-pyrazolo[3,4-d]pyrimidin-4(5H)-one, and 1-methyl-5-phenyl-6-((1-(pyridin-3-yl)ethyl)thio)-1H-pyrazolo[3,4-d]pyrimidin-4(5H)-one.

Additional ALDHis are described in U.S. Pat. No. 9,468,632, which is incorporated herein by reference in its entirety for its disclosure of ALDHis. In some embodiments, the inhibitors have the structure:

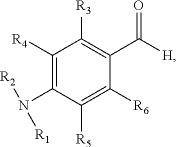

wherein R₁ and R₂ are independently or together, H, an alkyl, a cycloalkyl, together form a cycloalkyl or heterocyclic alkyl or aryl, an aryl, an alkenyl, a cycloalkyl, alkynl, or a substituted version of the aforementioned groups and R₃-R₆ are independently a halogen, H, an alkyl, a cycloalkyl, an aryl, an alkenyl, a cycloalkyl, alkynl, or a substituted version of the aforementioned groups. In some embodiments, the inhibitors have the structure:

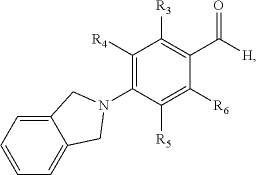

wherein and R₃-R₆ are independently a halogen, H, an alkyl, a cycloalkyl, an aryl, an alkenyl, a cycloalkyl, alkynl, or a substituted version of the aforementioned groups. In some embodiments, the compounds have the structure

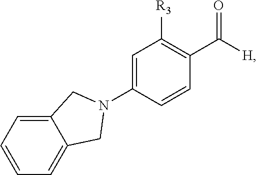

wherein R₃ is a halogen, H, an alkyl, a cycloalkyl, an aryl, an alkenyl, a cycloalkyl, alkynl, or a substituted version of the aforementioned groups. In some embodiments, the inhibitor is, for example,

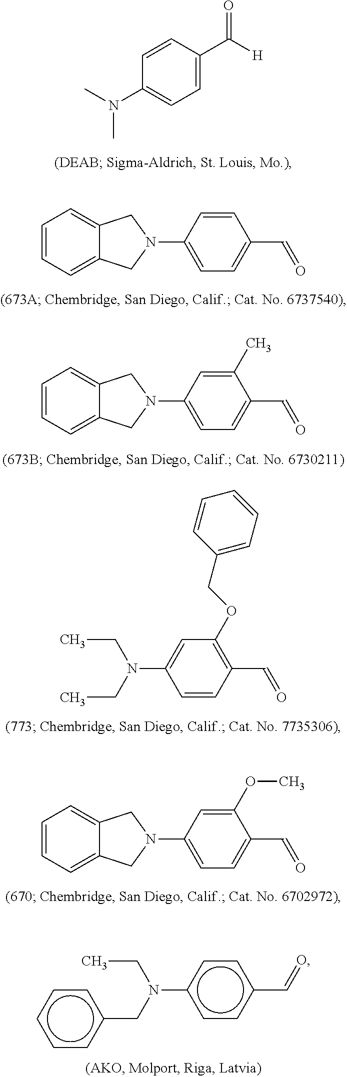

or one or more of,

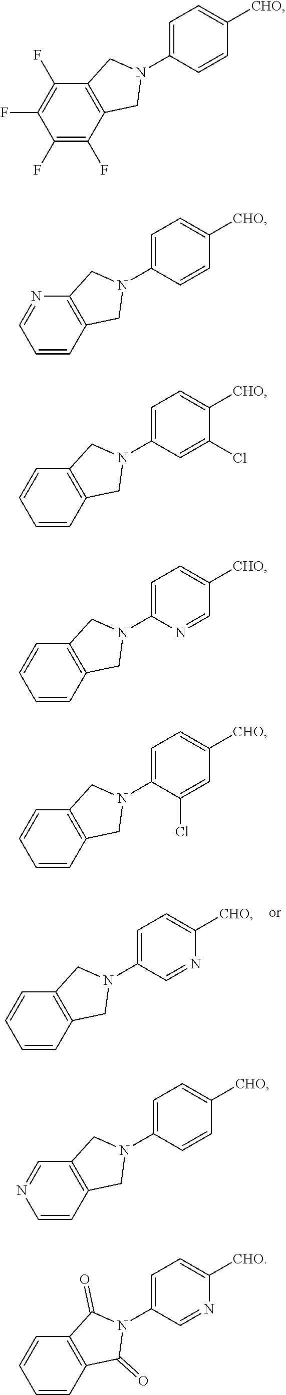

In one example, the compound is 4-(1,3-dihydroisoindol-2-yl)-3-methoxybenzaldehyde (also, “960”):

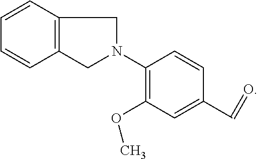 [SMILES: COc1cc(C=O)ccc1N1Cc2ccccc2C1]

In some embodiments, the inhibitor is a metabolite of any of the aforementioned compounds (e.g.,

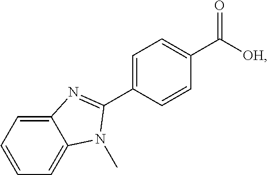 [SMILES: Cn1c(-c2ccc(C(=O)O)cc2)nc2ccccc21]

or a variant thereof). In some embodiments, the inhibitor is a prodrug and the metabolite of the compound is the active agent. In some embodiments, the compound is modified with a label to allow the compound to function as an imaging agent (e.g., radioactively labeled).

Suitable formulations, dosages, and dosing regimens can be optimized for each given ALDHi may be determined according to standard medical and pharmaceutical practice.

### Example 1

Mice and Cell lines: C57BL/6 and LacZ-RARE mice were purchased from Jackson Laboratory. The mouse ovarian cancer cell lines 2F8 and 2F8Cis were generated previously in the Vlad lab. Human cells CAOV3 and OVCAR5 were seeded in complete Roswell Park Memorial Institute medium (cRPMI, RPMI 1640 with 10% FBS, 2 mM L-Glutamine and 1% Penicillin-Streptomycin, 1% non-essential amino acids and 50 μM 2-ME). Mouse cells including 2F8, 2F8Cis, mouse splenocytes, and bone marrow immune cells were seeded in complete Dulbecco's Modified Eagle's Medium (cDMEM, DMEM with 10% FBS, 2 mM L-Glutamine, 1% Penicillin-Streptomycin, 1% non-essential amino acids and 50 μM 2-ME). All cells were cultured in humidified incubator at 37° C. with 5% CO₂. All experiments were conducted according to Institutional Animal Care and Use Committee (ACUC)-approved protocol and in accordance with NIH guidelines.

Drugs and treatments: Exemplary first- and second-generation ALDHis (673A (also referred to as “673” in the Figures) and 548 (262548, as in FIGS. 2B and 2C)) were obtained. All trans retinoic acid (ATRA, also known as Tretinoin) was purchased from the Peprotech company. Dimethyl sulfoxide (DMSO) (Sigma) was used for the reconstitution of these drugs as well as for the control treatment. The dosage of drug for treatment is 10 μM (673A) and 20 μM (548). The concentration for ATRA for treatment was 10 μM. The DMSO was added according to the volume of different drugs.

Splenocytes processing: 6-12 weeks old C57BL/6 or LacZ-RARE mice used. Spleens were collected at necropsy, rinsed twice with 1×PBS and then transferred into 10 cm Petri dish containing DMEM. Spleens were dissected into small pieces, passed through 70 micro filter and mechanically disrupted with a syringe plunger, and the resulting single cell suspension of splenocytes was collected into a 50 ml Falcon tube. Splenocytes were washed twice after the red blood cell lysis. Cells were counted using the Trypan blue exclusion method and kept on ice until further processing.

Splenocytes polyclonal stimulation: 24-well flat bottom cell culture plate (Thermo Fisher, Cat #142475) were precoated with 10 mg/ml of anti-CD3 antibody (BD, clone: 145-2C11, Cat #553057) diluted in 250 μl of 1×PBS either at 4° C. overnight or 2 hrs. at 37° C. After coating, wells were washed with 1×PBS before cells plating. 2 ml of 1×10⁶/ml splenocytes were suspended in cDMEM medium containing 2-ME and added to each well. 2 mg/ml anti-CD28 (BD, clone: 37.51, 553294) and 10 U/ml IL-2 (Peprotech) were added to each well and incubated at 37° C. for 3 days

Naïve CD4+ T cell selection and T cell subsets culture: Naïve CD4+ T cell were isolated from the splenocyte suspension using the EasySep™ Mouse Naïve CD4+ T Cell Isolation Kit (Negative Isolation, Cat #19765). Isolated cells were plated at the concentration of 5×10⁵/ml/well in a 48-well plate (Thermo Fisher) which were precoated with 10 mg/ml of anti-CD3 antibody (clone 145-2C11). 2 mg/ml of soluble anti-CD28 antibody was added to each well. Following reagents were added for each condition: Th0, human IL-2 (20 U/mL); Th1, human IL-2 (50 U/mL), murine IL-12 (Peprotech, 5 ng/mL), murine INF-γ (Sigma-Aldrich, 10 ng/mL), and anti-mouse IL-4 (e-Bioscience Clone: 11B11, 10 ug/mL); Th2, human IL-2 (20 U/mL), murine IL-4 (10 ng/mL), and anti-mouse INF-γ (e-Bioscience clone: R4-6A2, 10 ug/mL); Treg, human IL-2 (20 U/mL) and murine TGF-β (R&D, 5 ng/mL); Th17, murine IL-6 (R&D, 20 ng/mL), murine IL-23 (R&D, 10 ng/mL), murine TGF-β (R&D, 2 ng/mL), anti-mouse INF-γ (e-Bioscience, 10 ug/mL), anti-mouse IL-4 (e-Bioscience, 10 ug/mL), and anti-mouse IL-2 (e-Bioscience Clone: JES6-1A12, 10 ug/mL). Cells were harvested after 4-6 days and sorted with Flow Cytometry (LSR II BD Bioscience).

T cell subsets co-culture experiment: To further explore the function of the differentiated Th1, Th2, Treg and Th17 cells, co-culture experiment was carried out. Suspended mouse splenocytes (2×10⁶ in 2 mL cDMEM/well) were seeded in 24-well plate precoated with anti-mouse CD3 (10 ng/mL) and supplemented with anti-mouse CD28 (2 mg/mL) and human IL-2 (20 U/mL). Differentiated Th subsets were added to the splenocytes as 1:1 ratio to that of CD8+ cells numbers of fresh splenocytes. Co-cultured cells were collected after 2-3 days and sorted with Flow Cytometry.

Bone marrow processing and dendritic cell maturation: Bone marrow was extracted from mouse femurs and tibias and passed through a 70-micron filter. Red blood cells were lysed and remaining cells were collected after centrifugation. Resulting cells were plated as 4 ml per well in a 6-well plate at a density of 4×10⁶/mL in an ultra-low attachment plate (Corning) and cultured in AIM-V medium (Thermo Fisher). To obtain immature DCs, cells were cultured in granulocyte-macrophage colony stimulating factor GM-CSF (Thermo Fisher, 10 ng/ml). Half of the culture media containing GM-CSF was replenished mid-protocol, after 3 days of culture.

DC maturation: At day 7, immature DCs were collected and plated at the density of 3-4×10⁶/mL and treated with GM-CSF (10 ug/mL), LPS (250 ng/mL) (Sigma-Aldrich), IL-4 (1000 U/ML) (Sigma-Aldrich), IFN-γ (1000 U/mL) (Sigma-Aldrich), and polyinosinic:polycytidylic acid (poly I:C) (20 μg/mL) (sigma-Aldrich). Cells were matured in this cocktail overnight and harvested next day. Cells were profiled using flow cytometry.

Preparation of ID8 cells for antigen or extract to feed to DC cells: ID8 mouse ovarian cancer cells were harvested with harvest media (2 mM EDTA in 1×DPBS) as follow: Remove culture media and wash cells with 10 ml of harvest media and discard the media. Add fresh pre-warm harvest media and incubate it at 37° C. for 10-15 min. Dislodge cells by pipetting and collect them in a sterile centrifuge tube and wash 2× with 1×DPBS. Count cells and adjust to 1 M/ml in DPBS and plate in a 6 well plate, 1 ml per well and treat with UV light for 25 min in a laminar flow hood with UV light turn on. Collect cells by washing wells with DPBS and centrifuge at 800 g for 8 min to collect all cells and apoptotic bodies. Resuspend cell in cRPMI at 1M/ml and freeze thaw in Liq. N₂ and 37° C. water bath to kill all cells before adding to DC cells. Repeat freeze thaw 4 times and test a sample with Trypan blue to ensure all cells are dead. Store on ice until use.

Flow cytometry (Surface staining): All cells were washed and suspended in FACS buffer (2% BSA in 1×PBS) and incubated with antibodies for 30 minutes on ice in dark. For splenocytes, Anti-mouse CD3 (CD, clone: 145-2C11), Anti-mouse CD4 (BD, clone: RM4-5), Anti-mouse CD8 (BD, clone: 53-6.7), Anti-mouse CD25 (BD, clone: PC61 (RUO)), Anti-mouse CD11b (BioLegend, clone: M1/70), Anti-mouse CD274 (PD-L1) (BD, clone: MIH5), Anti-mouse CD279(PD-1) (BD, J43) and Anti-mouse CD366 (Tim3) (BD, clone: 5D12/Tim-3) antibodies were used for staining. For DCs maturation sorting, Anti-mouse MHC II (1-Ab) (BioLegend, clone: AF6-120.1), Anti-mouse CD11c (BioLegend, clone: N418), Anti-CD80 (BD, clone: 16-10A1), and Anti-CD86 (BD, clone: GL1) antibodies were used.

Flow cytometry (Intracellular staining): For Th subsets cells, intracellular (IC) staining was performed after the cell surface staining. Cells were fixed post surface staining for 20 minutes at room temperature in IC fixation buffer (invitrogen). Post fixation, cells were washed with 1× permeabilization buffer (PB) (Invitrogen, 10×) and incubated with antibodies diluted in 1×PB buffer. Anti-mouse INF-γ (Th1, BD, XMG1.2), Anti-mouse IL-4 (Th2, BD, 11 B11), Anti-mouse Mo/Rt (Treg, FOXP3) (Invitrogen, clone: FJK-16s), and Anti-mouse IL-17 (Th17, BD, monoclonal) antibodies were incubated for 30 minutes on ice in dark. Cells were washes twice with 1× permeabilization buffer before sorting.

Western Blotting: Cells were washed with 1×PBS and lysed in RIPA buffer containing Halt Protease and Phosphatase inhibitor cocktail. The resulting cell extracts were quantitated with BSA protein quantitation. Equal quantities of proteins were taken and boiled with SDS buffer and resolved on SDS-PAGE gel and transferred to nitrocellulose membrane. Membranes were blocked in 5% milk protein in 1×TBS and incubated with various primary and secondary antibodies. Resulting membranes were treated with enhanced ECL (Thermo Scientific) and detected with ChemiDoc XRS system (Bio-Rad) to acquire images.

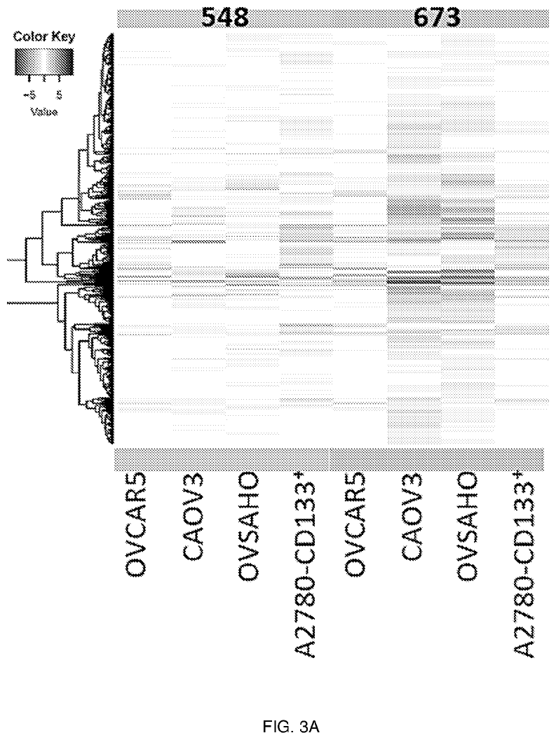

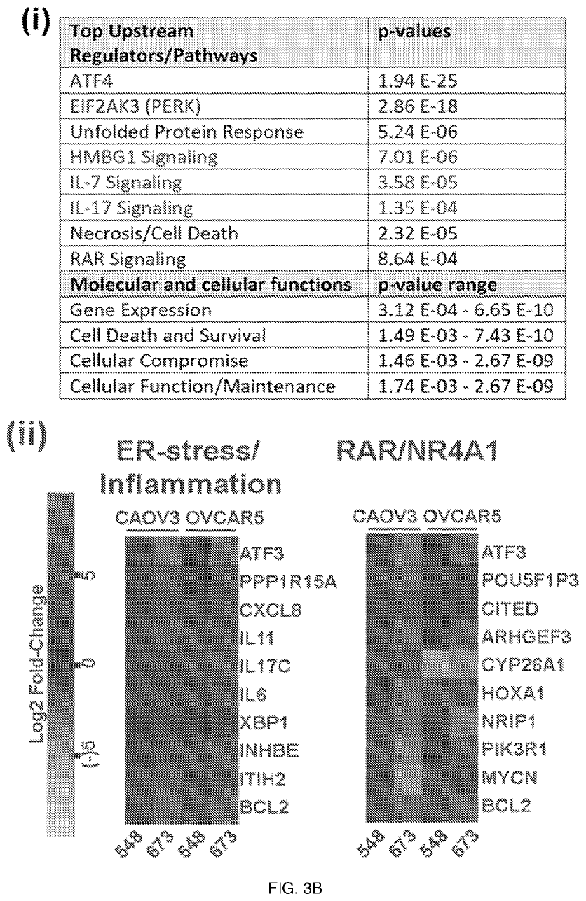

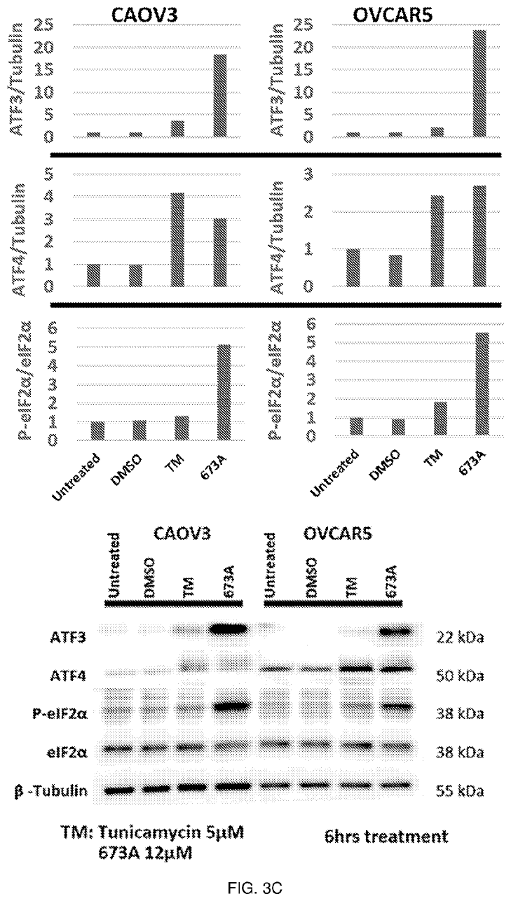

RNA-Seq was performed on four human ovarian cancer cell lines (A2780, CAOV3, OVCAR5, OVSAHO) treated for 8 hours with ALDHi 673A or 548. Heatmap in FIG. 3A shows differentially expressed genes that are similarly upregulated (blue in original) or downregulated (red in original) in response to ALDHi treatment. Using Ingenuity Pathway Analysis (IPA) of genes which were differentially expressed (p<0.01) in both 673A- and 548-treated cells, we identified statistically significant changes in ER stress unfolded protein response and regulators of the ER stress response, such as ATF4 and EIF2aK3 (PERK), inflammatory pathways, including HMBG1, IL-7, and IL-17 signaling, cell death, survival, maintenance, and gene transcription-RAR signaling (FIG. 3B). Heat map analysis (using data from two of the four lines tested, CAOV3 and OVCAR5) demonstrates log 2 fold change vs. control of several of the ER stress and inflammation-linked genes (ATF3, XBP1, IL6, IL17C, etc.) and known RA targets (CYP26A1, POUF51 P3, and HOXA2), FIG. 3B). In line with gene expression data, we confirmed that at protein level, ALDHi treatment leads to very strong induction of the ER stress driving ATF3 and ATF4 transcription factor and a 5-X increase in p-eIF2a levels in two separate cell lines (western blot results in FIG. 3C). Kroemer and colleagues have reported that pre-mortem ER stress and eIF2a phosphorylation is “a quintessential hallmark” of immunogenic cell death (ICD) in cancer (Bezu, L., et al., eIF2a phosphorylation: A hallmark of immunogenic cell death. OncoImmunology, 2018. 7(6): p. e1431089).

Overall, using two different ALDHi compounds tested on four different human ovarian cancer cell lines we were able to identify commonly triggered genes and biological pathways modified by ALDHi and to demonstrate that ALDHi induce a type of death (ICD) in cancer cells which can stimulate anti-tumor immunity.

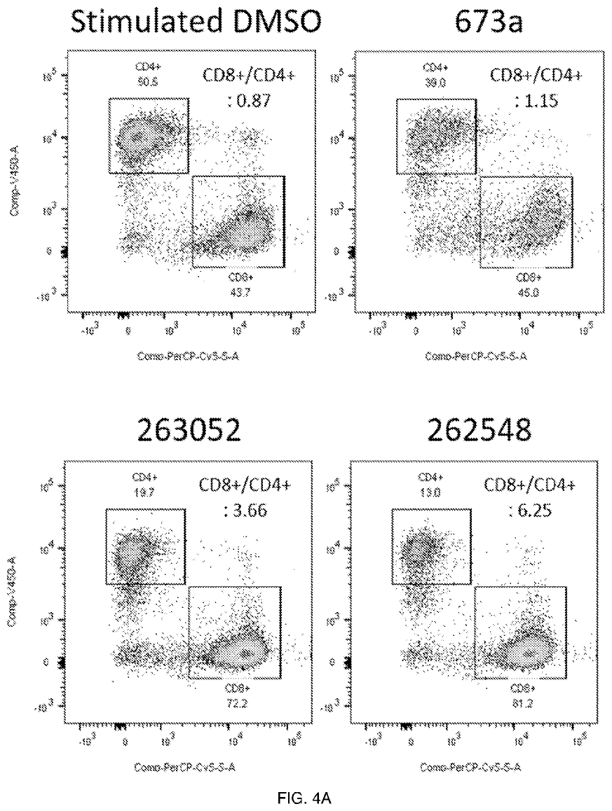

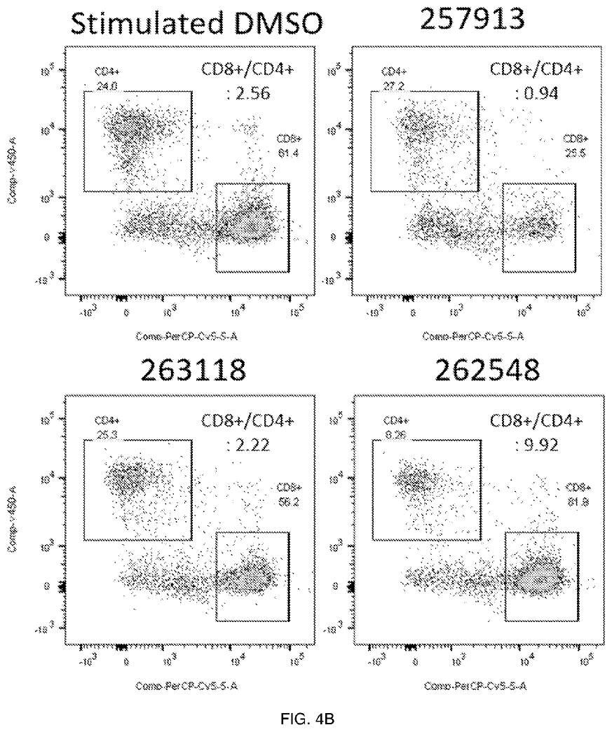

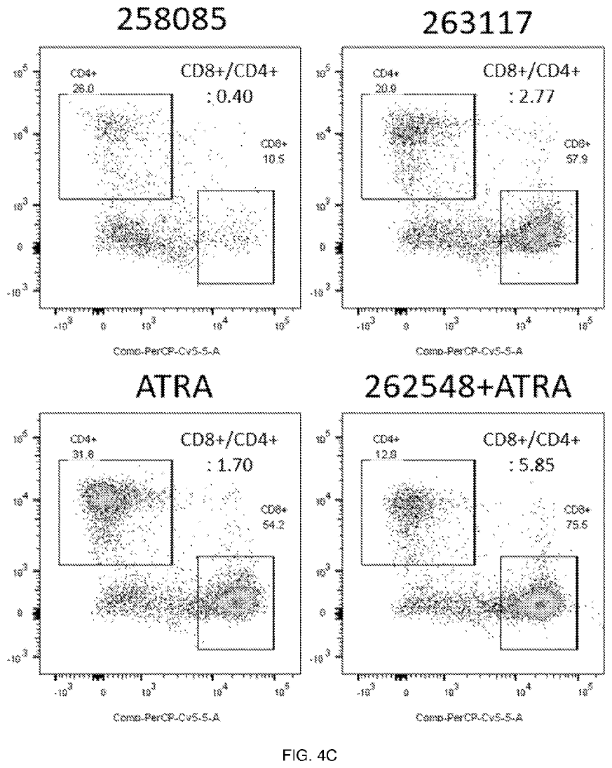

FIGS. 4A-4C—Polyclonal stimulation (using anti-CD3/CD28) of murine T cells (from spleens), with or without ADLHi. In the absence of ALDHi (DMSO only, used as control), stimulation expands both CD4 and CD8 T cells and the ratio of CD8 to CD4 T cells is less than 1 (as expected in the spleen). However, exposure of T cells to some of the ALDHi compounds during polyclonal stimulation, strongly favors proliferation of CD8 T cells, leading to CD8/CD4 ratios that are higher than 1. Drugs like 262548 (or briefly, 548) triggers a CD8/CD4 ratio of almost 10 (panel B). The effect is lowered (to a ratio of 6) when ALDHi is administered together with ATRA (which is expected to antagonize the efficacy of ALDHi). Increases in CD8/CD4 ratios are more modest in response to ALDHi 263052, 263117, 263118 while others (257913) do not seem to trigger significant changes over DMSO control.

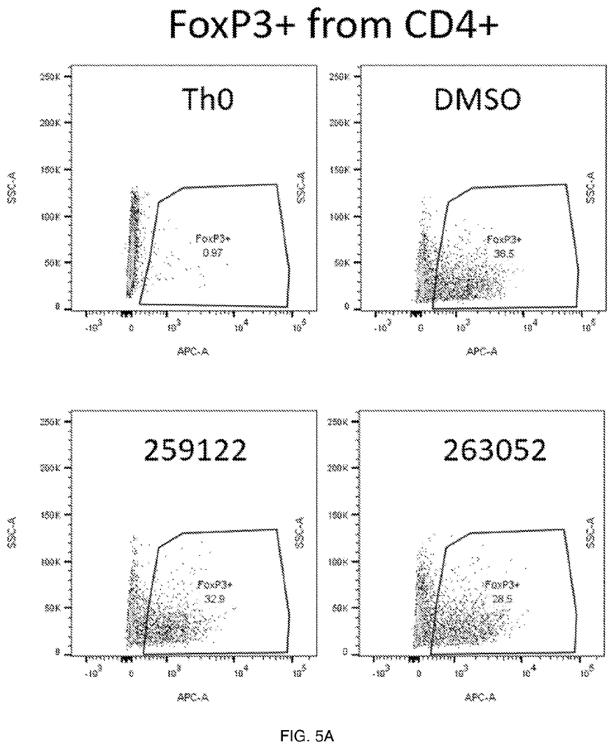

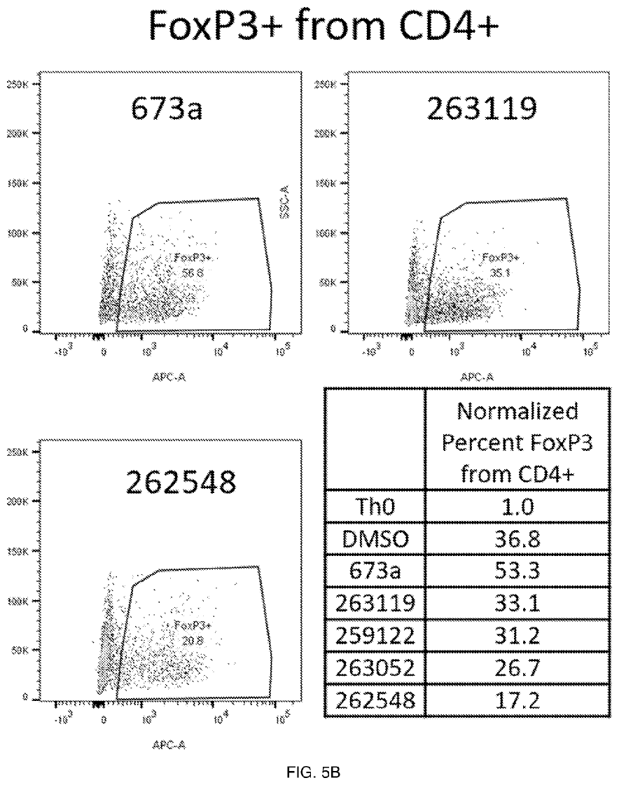

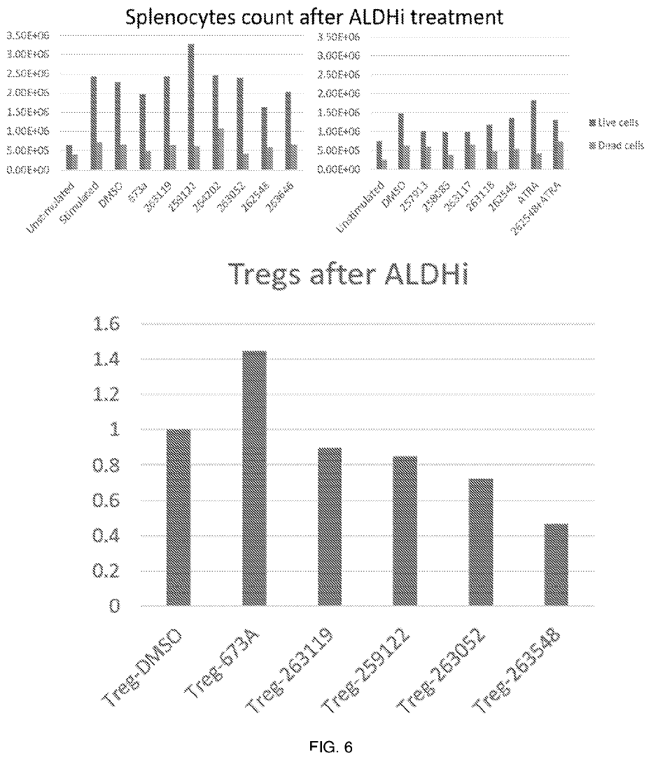

FIGS. 5A, 5B, and FIG. 6—Murine naïve CD4 T cells were isolated from spleens and subjected to Treg induction protocol, in the presence of five different ALDHi compounds. DMSO-exposed cells were used as control. At the end of protocol the cells were stained intracellularly for FoxP3. Percentage of Tregs are shown. These results demonstrate that some compounds (especially) 262548 can effectively inhibit differentiation into Tregs.

### Example 2

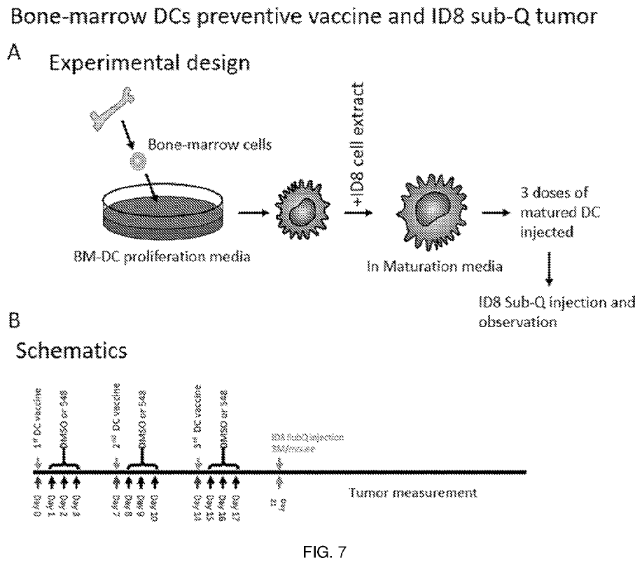

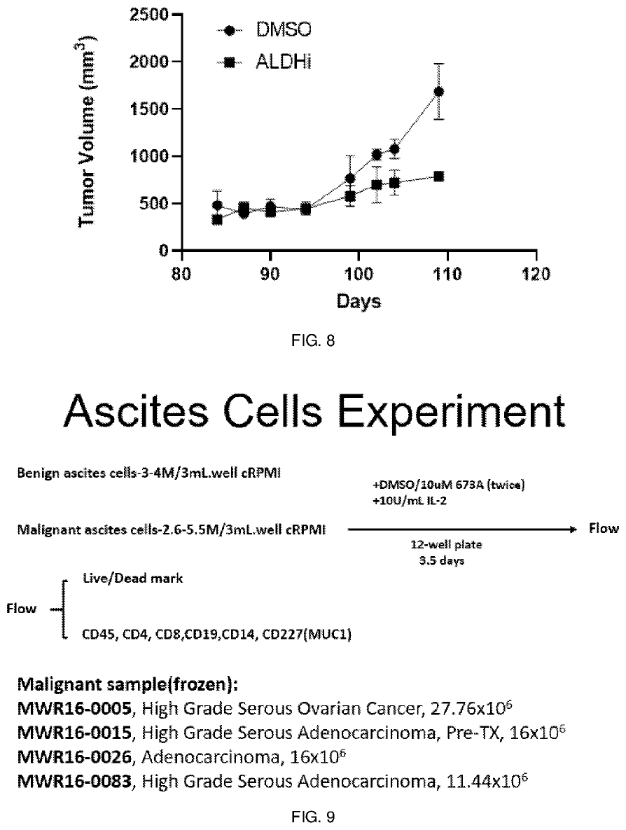

FIG. 7—In this example, DC were grown from bone marrow precursors in DC growth medium for 6 days. Immature DCs were exposed to cell lysate from ID8 cells and then matured in the presence of DC1 maturation media. Tumor antigen loaded, matured DC1 cells were used as vaccine (150-200 k cells/vaccine), injected in the right flank three times, at one week interval (days 0, 7 and 14). The vaccine was followed by daily IP injection of ALDHi (548) or DMSO control=for three consecutive days in each treatment cycle. Four days after completion of cycle 3 (day 21), mice were challenged with 3 million ID8 tumor cells, injected subcutaneously in the right flank (see, FIG. 7, outlining the experiment schematically). Tumor volume was measured at various time points, and results were graphed, as shown in FIG. 8. A significant reduction in tumor growth was seen in mice to which the ALDHi 548 was administered in the days following DC therapy, indicating that the ALDHi increases antigen-specific immune response in animals administered ex vivo-generated DCs loaded with tumor antigens. Other dendritic-cell immunotherapies that rely on antigen recognition, and which rely on the generation of an immune response to an antigen, are expected to benefit from use of an ALDHi adjuvant therapy.

### Example 3—Ascites Cell Experiment

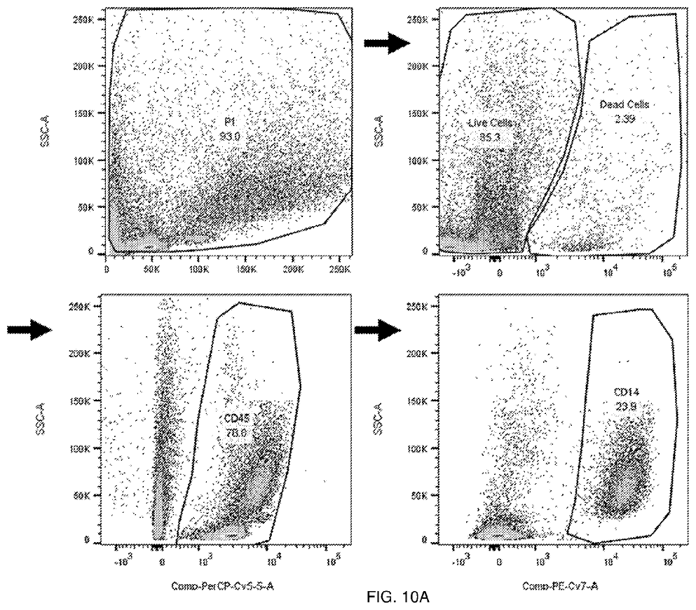

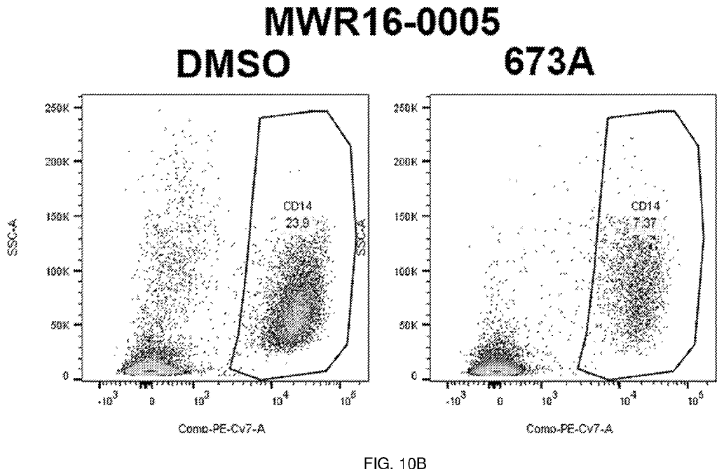

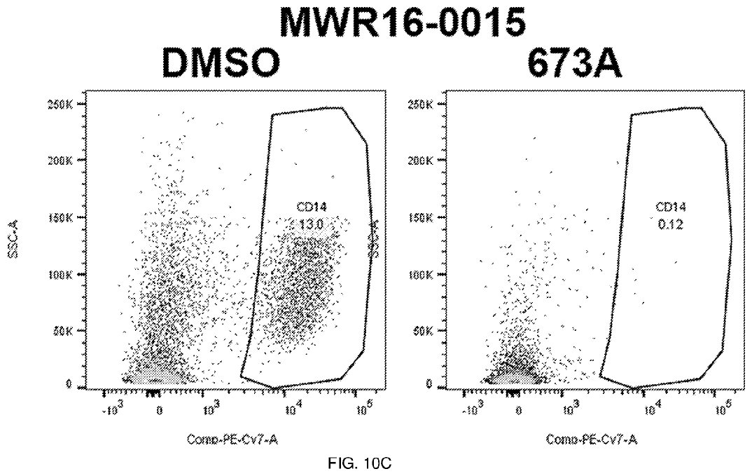

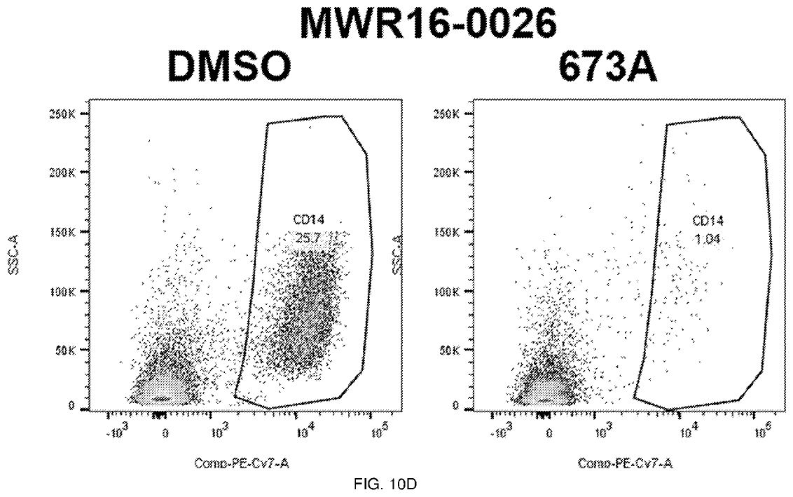

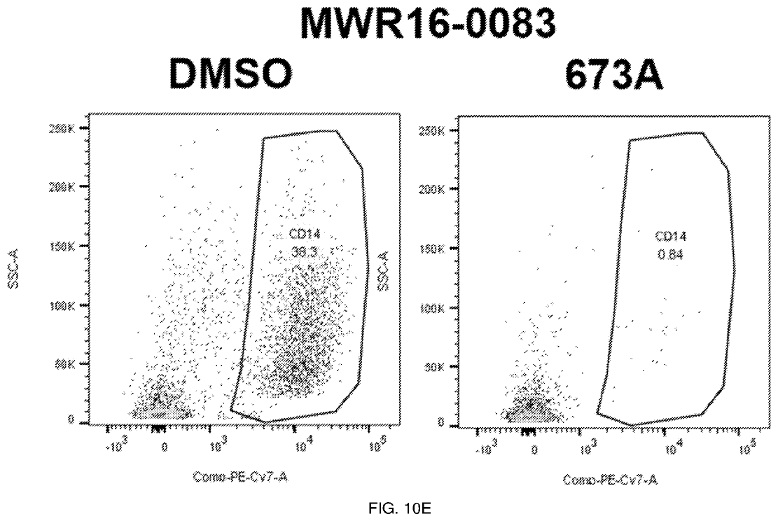

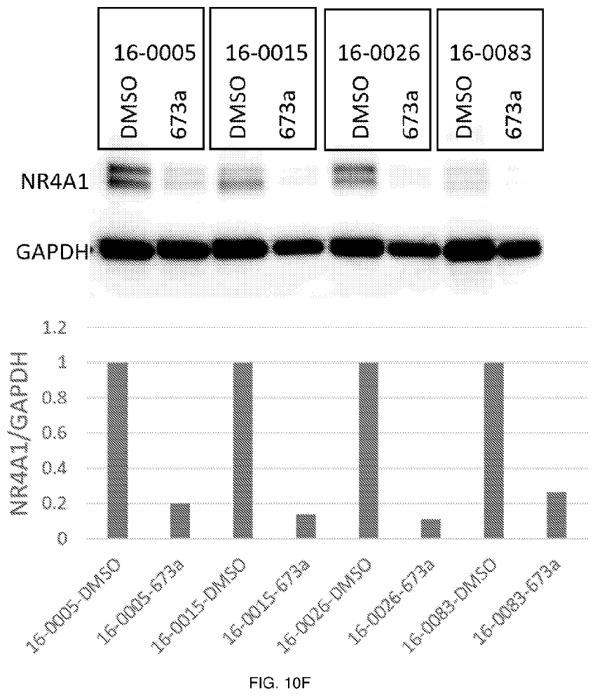

FIGS. 10A-10F—Ascites cells from four different ovarian cancer patients were plated using the conditions outlined in FIG. 9. Cells were treated with either 673A (10 uM) or DMSO control for 3 days, after which flow cytometry was performed. FIG. 10A shows how gating was performed and how within the live cell population (using Live/Dead dye) the cells of interest were identified. CD45 identifies all immune cells while CD14 is specific for monocyte/macrophages. The results show a significant decrease in ascites CD14 positive cells exposed to ALDHi (but not DMSO) (FIGS. 10B-10E), suggesting that ALDHi is toxic to ascites resident macrophages. Following treatment, protein was extracted from remaining cells and western blot was performed (panel C, FIG. 10F). In line with our hypothesis, NR4A1 was lowered by ALDHi treatment. Ascites macrophages have mostly tumor promoting functions and these results suggest that ALDHi may exert a toxic effect on this type of cells.

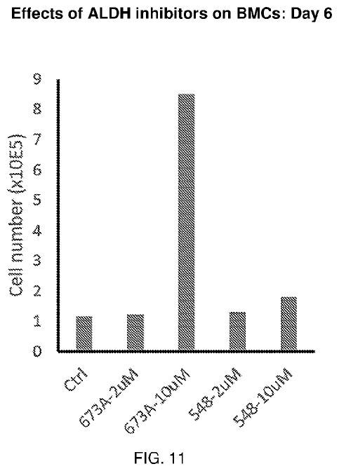

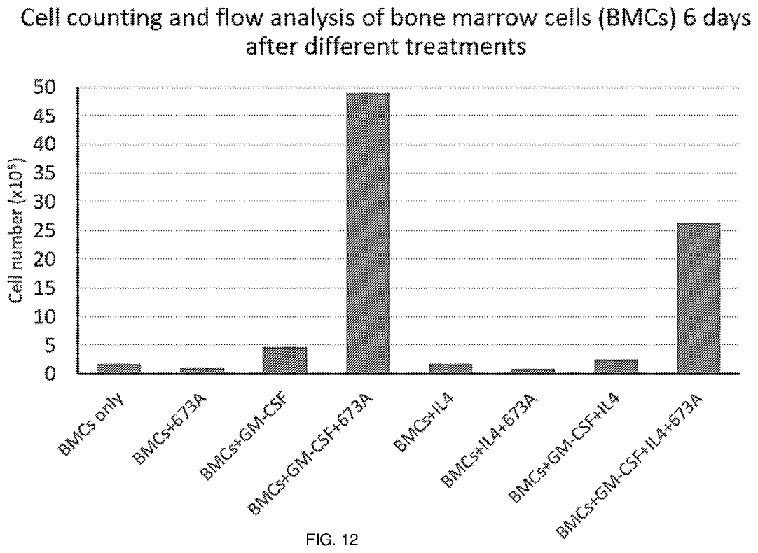

FIGS. 11 and 12—Generation of DC from bone marrow precursors in the presence of two different ALDHi (673A and 548) at two different concentrations (2 μM and 10 μM, respectively) shows a significant (nine fold) expansion in the presence of 673A (10 uM), compared to DMSO control. These results suggest that some ALDHi may possess the capacity to support the expansion (in vitro and potentially in vivo) of dendritic cells.

Having described this invention above, it will be understood to those of ordinary skill in the art that the same can be performed within a wide and equivalent range of conditions, formulations and other parameters without affecting the scope of the invention or any embodiment thereof. Any document incorporated herein by reference is only done so to the extent of its technical disclosure and to the extent it is consistent with the present document and the disclosure provided herein.

1. A method of expanding bone marrow cells and/or dendritic cell populations, comprising culturing bone-marrow cells and/or dendritic cells in culture medium comprising an amount of an Aldehyde dehydrogenase-1A enzyme inhibitor (ALDHi) effective to increase a population of the bone-marrow cells and/or dendritic cells as compared to an increase in the population of the bone-marrow cells and/or dendritic cells grown in the same culture medium and conditions without the ALDHi in the culture medium.

2. The method of claim 1, wherein the ALDHi has the 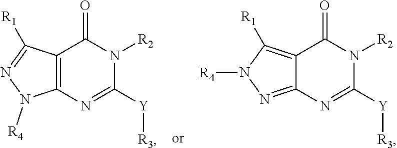
including pharmaceutically acceptable salts, solvates, and/or prodrugs thereof,
wherein:
R₁ is H or C₁-C₃ alkyl (substituted or non-substituted);
R₂ is H, C₀-C₃-alkyl-aryl or C₀-C₃-alkyl-heteroaryl, wherein the alkyls can be independently substituted or non-substituted, such as CH₃, or selected from one of the following: 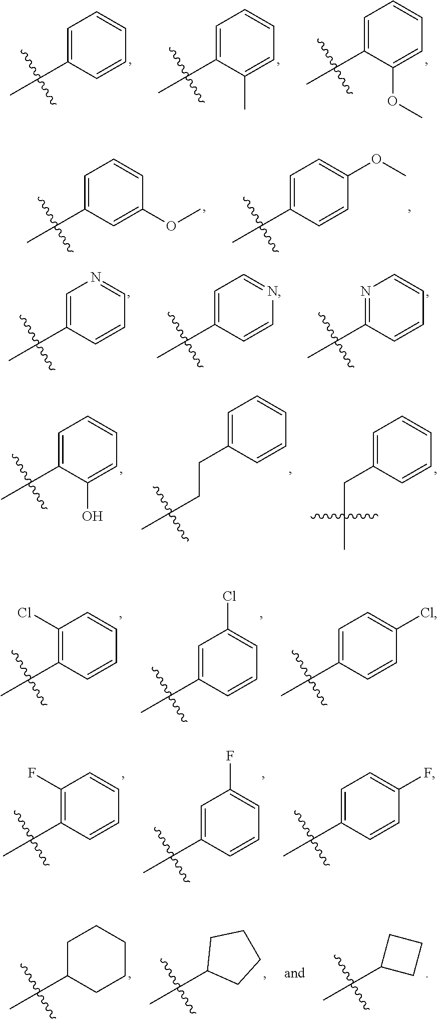
Y is a linker, and optionally an alkyl, alkenyl, heteroalkyl, or heteroalkenyl, where the hetero atom is S, N, or O that is optionally substituted;
R₃ is H, C₀-C₈ alkyl, C₀-C₂-alkyl-aryl or C₀-C₂-alkyl-heteroaryl, wherein the alkyls are independently substituted or non-substituted; and
R₄ is H, C₁-C₆ alkyl or cycloalkyl, optionally substituted, wherein one or more carbons may be replaced by oxygen, and wherein the alkyls are independently substituted or non-substituted, such as a structure of one of the following:

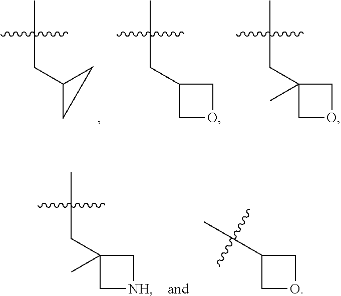

3-8. (canceled)

9. The method of claim 1, wherein the ALDHi is selected from one of the following, or a pharmaceutically acceptable salt thereof: 6-((3-fluorobenzyl)thio)-5-(o-tolyl)-1H-pyrazolo[3,4-d]pyrimidin-4(5H)-one, 6-((3-fluorobenzyl)thio)-5-phenyl-1H-pyrazolo[3,4-d]pyrimidin-4(5H)-one, 6-((3-fluorobenzyl)thio)-1-methyl-5-(o-tolyl)-1H-pyrazolo[3,4-d]pyrimidin-4(5H)-one, 6-((3-fluorobenzyl)thio)-2-methyl-5-(o-tolyl)-2H-pyrazolo[3,4-d]pyrimidin-4(5H)-one, 1-ethyl-6-((3-fluorobenzyl)thio)-5-(o-tolyl)-1H-pyrazolo[3,4-d]pyrimidin-4(5H)-one, 2-ethyl-6-((3-fluorobenzyl)thio)-5-(o-tolyl)-2H-pyrazolo[3,4-d]pyrimidin-4(5H)-one, 1-(cyclopropylmethyl)-6-((3-fluorobenzyl)thio)-5-(o-tolyl)-1H-pyrazolo[3,4-d]pyrimidin-4(5H)-one, 2-(cyclopropylmethyl)-6-((3-fluorobenzyl)thio)-5-(o-tolyl)-2H-pyrazolo[3,4-d]pyrimidin-4(5H)-one, 6-((3-fluorobenzyl)thio)-1-(oxetan-3-yl)-5-(o-tolyl)-1H-pyrazolo[3,4-d]pyrimidin-4(5H)-one, 6-((3-fluorobenzyl)thio)-2-(oxetan-3-yl)-5-(o-tolyl)-2H-pyrazolol[3,4-d]pyrimidin-4(5H)-one, 6-((3-fluorobenzyl)thio)-1-(oxetan-3-ylmethyl)-5-(o-tolyl)-1H-pyrazolol[3,4-d]pyrimidin-4(5H)-one, 6-((3-fluorobenzyl)thio)-2-(oxetan-3-ylmethyl)-5-(o-tolyl)-2H-pyrazolo[3,4-d]pyrimidin-4(5H)-one, 6-((3-fluorobenzyl)thio)-2-methyl-5-phenyl-2H-pyrazolo[3,4-d]pyrimidin-4(5H)-one, 1-ethyl-6-((3-fluorobenzyl)thio)-5-phenyl-1H-pyrazolo[3,4-d]pyrimidin-4(5H)-one, 2-ethyl-6-((3-fluorobenzyl)thio)-5-phenyl-2H-pyrazolo[3,4-d]pyrimidin-4(5H)-one, 1-(cyclopropylmethyl)-6-((3-fluorobenzyl)thio)-5-phenyl-1H-pyrazolo[3,4-d]pyrimidin-4(5H)-one, 2-(cyclopropylmethyl)-6-((3-fluorobenzyl)thio)-5-phenyl-2H-pyrazolo[3,4-d]pyrimidin-4(5H)-one, 6-((3-fluorobenzyl)thio)-1-(oxetan-3-yl)-5-phenyl-1H-pyrazolo[3,4-d]pyrimidin-4(5H)-one, 6-((3-fluorobenzyl)thio)-2-(oxetan-3-yl)-5-phenyl-2H-pyrazolo[3,4-d]pyrimidin-4(5H)-one, 6-((3-fluorobenzyl)thio)-1-(oxetan-3-ylmethyl)-5-phenyl-1H-pyrazolo[3,4-d]pyrimidin-4(5H)-one, 6-((3-fluorobenzyl)thio)-2-(oxetan-3-ylmethyl)-5-phenyl-2H-pyrazolo[3,4-d]pyrimidin-4(5H)-one,
6-((3-fluorobenzyl)amino)-5-(o-tolyl)-1H-pyrazolo[3,4-d]pyrimidin-4(5H)-one, 6-((3-fluorophenethyl)amino)-1-methyl-5-(o-tolyl)-1H-pyrazolo[3,4-d]pyrimidin-4(5H)-one, 6-((3-fluorobenzyl)oxy)-1-methyl-5-(o-tolyl)-1H-pyrazolo[3,4-d]pyrimidin-4(5H)-one, 6-((3-fluorophenoxy)methyl)-1-methyl-5-phenyl-1H-pyrazolo[3,4-d]pyrimidin-4(5H)-one, 6-(((3-fluorobenzyl)amino)methyl)-1-methyl-5-phenyl-1H-pyrazolo[3,4-d]pyrimidin-4(5H)-one, 6-((3-fluorobenzyl)thio)-1-methyl-5-phenyl-1H-pyrazolo[3,4-d]pyrimidin-4(5H)-one, 6-((3-fluorobenzyl)thio)-5-(2-methoxyphenyl)-1-methyl-1H-pyrazolo[3,4-d]pyrimidin-4(5H)-one, 6-((3-fluorobenzyl)thio)-5-(3-methoxyphenyl)-1-methyl-1H-pyrazolo[3,4-d]pyrimidin-4(5H)-one, 6-((3-fluorobenzyl)thio)-5-(4-methoxyphenyl)-1-methyl-1H-pyrazolo[3,4-d]pyrimidin-4(5H)-one 6-((3-fluorobenzyl)thio)-1-methyl-5-(pyridin-3-yl)-1H-pyrazolo[3,4-d]pyrimidin-4(5H)-one, 6-((3-fluorobenzyl)thio)-1-methyl-5-(pyridin-4-yl)-1H-pyrazolo[3,4-d]pyrimidin-4(5H)-one, 6-((3-fluorobenzyl)thio)-5-(2-hydroxyphenyl)-1-methyl-1H-pyrazolo[3,4-d]pyrimidin-4(5H)-one, 6-((3-fluorobenzyl)thio)-1-methyl-5-(pyridin-2-yl)-1H-pyrazolo[3,4-d]pyrimidin-4(5H)-one, 6-((3-fluorobenzyl)thio)-1-methyl-5-phenethyl-1H-pyrazolo[3,4-d]pyrimidin-4(5H)-one, 5-benzyl-6-((3-fluorobenzyl)thio)-1-methyl-1H-pyrazolo[3,4-d]pyrimidin-4(5H)-one, 6-((3-fluorobenzyl)thio)-1,5-dimethyl-1H-pyrazolo[3,4-d]pyrimidin-4(5H)-one, 6-(isopentylthio)-1-methyl-5-phenyl-1H-pyrazolo[3,4-d]pyrimidin-4(5H)-one, 6-((3-fluorobenzyl)thio)-3-methyl-5-(o-tolyl)-2H-pyrazolol[3,4-d]pyrimidin-4(5H)-one, 6-(benzo[b]thiophen-2-ylmethoxy)-1-methyl-5-(o-tolyl)-1H-pyrazolo[3,4-d]pyrimidin-4(5H)-one, 5-(2-chlorophenyl)-6-(3-fluorobenzylthio)-1-methyl-1H-pyrazolo[3,4-d]pyrimidin-4(5H)-one, 5-(2-chlorophenyl)-6-((3-fluorobenzyl)thio)-1-methyl-1H-pyrazolo[3,4-d]pyrimidin-4(5H)-one, 5-(3-chlorophenyl)-6-((3-fluorobenzyl)thio)-1-methyl-1H-pyrazolo[3,4-d]pyrimidin-4(5H)-one, 5-(4-chlorophenyl)-6-((3-fluorobenzyl)thio)-1-methyl-1H-pyrazolo[3,4-d]pyrimidin-4(5H)-one, 6-((3-fluorobenzyl)thio)-5-(2-fluorophenyl)-1-methyl-1H-pyrazolo[3,4-d]pyrimidin-4(5H)-one, 5-cyclohexyl-6-((3-fluorobenzyl)thio)-1-methyl-1H-pyrazolo[3,4-d]pyrimidin-4(5H)-one, 5-cyclopentyl-6-((3-fluorobenzyl)thio)-1-methyl-1H-pyrazolo[3,4-d]pyrimidin-4(5H)-one, 5-cyclobutyl-6-((3-fluorobenzyl)thio)-1-methyl-1H-pyrazolo[3,4-d]pyrimidin-4(5H)-one, (E)-6-(3-fluorostyryl)-1-methyl-5-phenyl-1H-pyrazolo[3,4-d]pyrimidin-4(5H)-one, 6-(3-fluorophenethyl)-1-methyl-5-phenyl-1H-pyrazolo[3,4-d]pyrimidin-4(5H)-one, 6-(((3-fluorophenyl)amino)methyl)-1-methyl-5-phenyl-1H-pyrazolo[3,4-d]pyrimidin-4(5H)-one, 6-(((3-fluorobenzyl)oxy)methyl)-1-methyl-5-phenyl-1H-pyrazolo[3,4-d]pyrimidin-4(5H)-one, 6-(((3-fluorophenyl)thio)methyl)-1-methyl-5-phenyl-1H-pyrazolo[3,4-d]pyrimidin-4(5H)-one, 1-methyl-6-((2-oxo-2-phenylethyl)thio)-5-phenyl-1H-pyrazolo[3,4-d]pyrimidin-4(5H)-one, 3-(((1-methyl-4-oxo-5-phenyl-4,5-dihydro-1H-pyrazolo[3,4-d]pyrimidin-6-yl)thio)methyl)benzonitrile, 6-((3-methoxybenzyl)thio)-1-methyl-5-phenyl-1H-pyrazolo[3,4-d]pyrimidin-4(5H)-one, 6-(benzylthio)-1-methyl-5-phenyl-1H-pyrazolo[3,4-d]pyrimidin-4(5H)-one, 1-methyl-5-phenyl-6-((3-(2,2,2-trifluoroacetyl)benzyl)thio)-1H-pyrazolo[3,4-d]pyrimidin-4(5H)-one, 3-(((1-methyl-4-oxo-5-phenyl-4,5-dihydro-1H-pyrazolo[3,4-d]pyrimidin-6-yl)thio)methyl)benzaldehyde, 4-(((1-methyl-4-oxo-5-phenyl-4,5-dihydro-1H-pyrazolo[3,4-d]pyrimidin-6-yl)thio)methyl)benzaldehyde, 6-((3-fluorobenzyl)oxy)-1-methyl-5-phenyl-1H-pyrazolo[3,4-d]pyrimidin-4(5H)-one, 1-methyl-5-phenyl-6-((1-phenylethyl)thio)-1H-pyrazolo[3,4-d]pyrimidin-4(5H)-one, 6-(((3-fluorophenyl)(methyl)amino)methyl)-1-methyl-5-phenyl-1H-pyrazolo[3,4-d]pyrimidin-4(5H)-one, 6-((3-fluorobenzyl)oxy)-2-(oxetan-3-ylmethyl)-5-phenyl-2H-pyrazolo[3,4-d]pyrimidin-4(5H)-one, 2-(oxetan-3-ylmethyl)-5-phenyl-6-((1-phenylethyl)thio)-2H-pyrazolo[3,4-d]pyrimidin-4(5H)-one, (R)-1-methyl-5-phenyl-6-((1-phenylethyl)thio)-1H-pyrazolo[3,4-d]pyrimidin-4(5H)-one, (S)-1-methyl-5-phenyl-6-((1-phenylethyl)thio)-1H-pyrazolo[3,4-d]pyrimidin-4(5H)-one, 1-methyl-5-phenyl-6-(1-phenylethoxy)-1H-pyrazolo[3,4-d]pyrimidin-4(5H)-one, 6-((3-fluorobenzyl)thio)-2-((3-methyloxetan-3-yl)methyl)-5-phenyl-2H-pyrazolo[3,4-d]pyrimidin-4(5H)-one, 3-((6-((3-fluorobenzyl)thio)-4-oxo-5-phenyl-4,5-dihydro-2H-pyrazolo[3,4-d]pyrimidin-2-yl)methyl)azetidin-1-ium sulfate, 1-methyl-5-phenyl-6-((1-phenylpropyl)thio)-1H-pyrazolo[3,4-d]pyrimidin-4(5H)-one, 6-((1-(3-fluorophenyl)ethyl)thio)-1-methyl-5-phenyl-1H-pyrazolo[3,4-d]pyrimidin-4(5H)-one, 6-(cyclohexylthio)-1-methyl-5-phenyl-1H-pyrazolo[3,4-d]pyrimidin-4(5H)-one, 1-(oxetan-3-yl)-5-phenyl-6-((1-phenylethyl)thio)-1H-pyrazolo[3,4-d]pyrimidin-4(5H)-one, 2-(oxetan-3-yl)-5-phenyl-6-((1-phenylethyl)thio)-2H-pyrazolo[3,4-d]pyrimidin-4(5H)-one, 1-methyl-6-((2-oxocyclopentyl)thio)-5-phenyl-1H-pyrazolo[3,4-d]pyrimidin-4(5H)-one, 1-methyl-6-((2-oxocyclohexyl)thio)-5-phenyl-1H-pyrazolo[3,4-d]pyrimidin-4(5H)-one, 1-methyl-5-phenyl-6-((2-phenylpropan-2-yl)thio)-1H-pyrazolo[3,4-d]pyrimidin-4(5H)-one, 6-((1-(3-hydroxyphenyl)ethyl)thio)-1-methyl-5-phenyl-1H-pyrazolo[3,4-d]pyrimidin-4(5H)-one, 1-methyl-5-phenyl-6-((1-(pyridin-2-yl)ethyl)thio)-1H-pyrazolo[3,4-d]pyrimidin-4(5H)-one, and 1-methyl-5-phenyl-6-((1-(pyridin-3-yl)ethyl)thio)-1H-pyrazolo[3,4-d]pyrimidin-4(5H)-one.

10. (canceled)

11. The method of claim 1, wherein the ALDHi is selected from 673A, 257913 (913), 258085 (085), 262548 (548), 263052 (052), 263117 (117), 263118 (118), 263119 (119), 259122 (122), 263646 (646), 264202 (202), or a pharmaceutically acceptable salt thereof.

12. The method of claim 1, wherein the ALDHi is 673A, or a pharmaceutically acceptable salt thereof.

13. The method of claim 1, wherein the ALDHi has the structure: 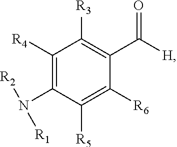
wherein R₁ and R₂ are independently or together, H, an alkyl, a cycloalkyl, together form a cycloalkyl or heterocyclic alkyl or aryl, an aryl, an alkenyl, a cycloalkyl, alkynl, or a substituted version of the aforementioned groups and R₃-R₆ are independently a halogen, H, an alkyl, a cycloalkyl, an aryl, an alkenyl, a cycloalkyl, alkynl, or a substituted version of the aforementioned groups, or a pharmaceutically acceptable salt thereof.

14. The method of claim 13, wherein the ALDHi has the structure: 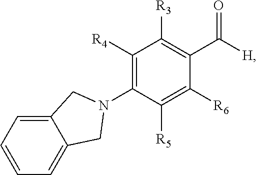
wherein and R₃-R₆ are independently a halogen, H, an alkyl, a cycloalkyl, an aryl, an alkenyl, a cycloalkyl, alkynl, or a substituted version of the aforementioned groups, or a pharmaceutically acceptable salt thereof.

15. The method of claim 1, wherein the ALDHi has a structure selected from: 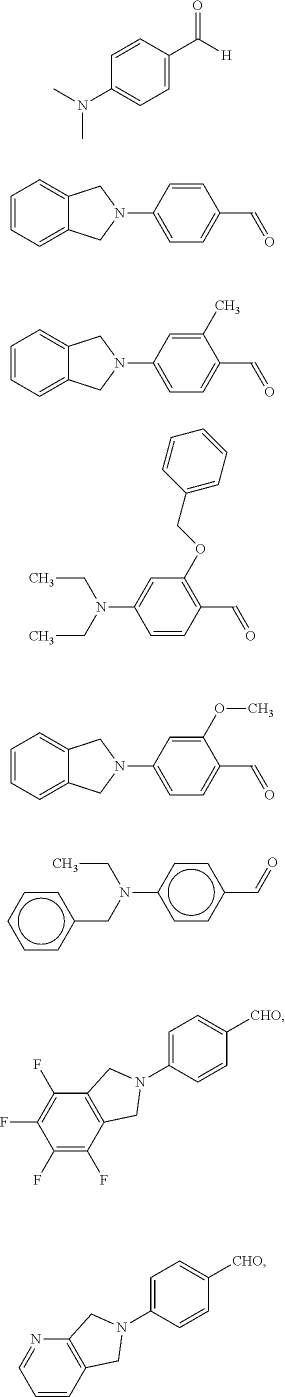 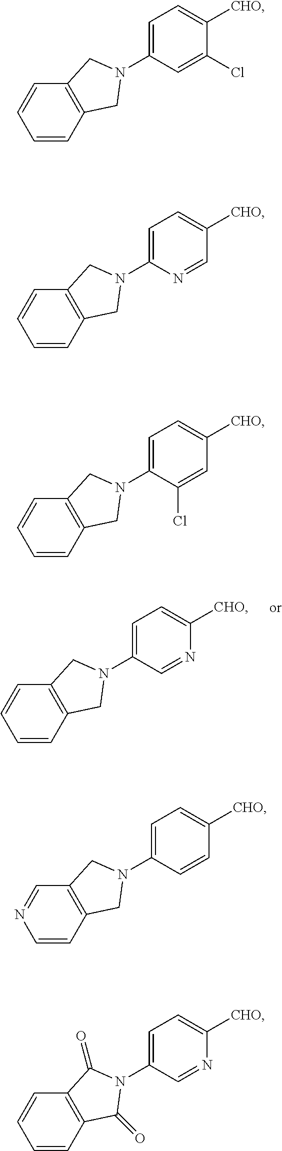 [SMILES: O=Cc1ccc(N2Cc3ccccc3C2)cc1Cl ; O=Cc1ccc(N2Cc3ccccc3C2)nc1 ; O=Cc1ccc(N2Cc3ccccc3C2)c(Cl)c1 ; O=Cc1ccc(N2Cc3ccccc3C2)cn1 ; O=Cc1ccc(N2Cc3ccncc3C2)cc1 ; O=Cc1ccc(N2C(=O)c3ccccc3C2=O)cn1]
or a pharmaceutically acceptable salt thereof.

16. The method of claim 1, wherein the ALDHi has a structure 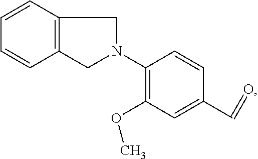 [SMILES: COc1cc(C=O)ccc1N1Cc2ccccc2C1]
or a pharmaceutically acceptable salt thereof.

17. An immunotherapy method for increasing an immune response to a pathogen or cancer cell antigen in a patient, comprising:
administering to a patient an immunotherapeutic agent for increasing an antigen-specific immune response in a patient; and
administering to the patient an adjuvant therapy to the administration of the immune cell, an amount of an aldehyde dehydrogenase-1A enzyme inhibitor (ALDHi) effective to reduce T_{REG} activity in the patient.

18. The method of claim 17, wherein the immunotherapeutic agent is an immune cell adapted to elicit an immune response to an antigen: an antigen-primed antigen-presenting (APC), such as a dendritic cell primed with an antigen a CAR-T cell; or a checkpoint inhibitor such as a PD1- or PDL1-targeting antibody (e.g., pembrolizumab, nivolumab, atezolizumab, or durvalumab).

19-23. (canceled)

24. The method of claim 17, wherein the ALDHi has the structure: 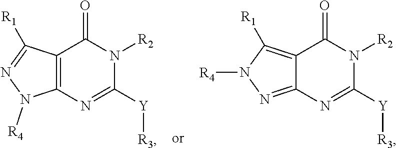
including pharmaceutically acceptable salts, solvates, and/or prodrugs thereof, wherein:
R₁ is H or C₁-C₃ alkyl (substituted or non-substituted)
R₂ is H, C₀-C₃-alkyl-aryl or C₀-C₃-alkyl-heteroaryl, wherein the alkyls can be independently substituted or non-substituted, such as CH₃, or selected from one of the following: 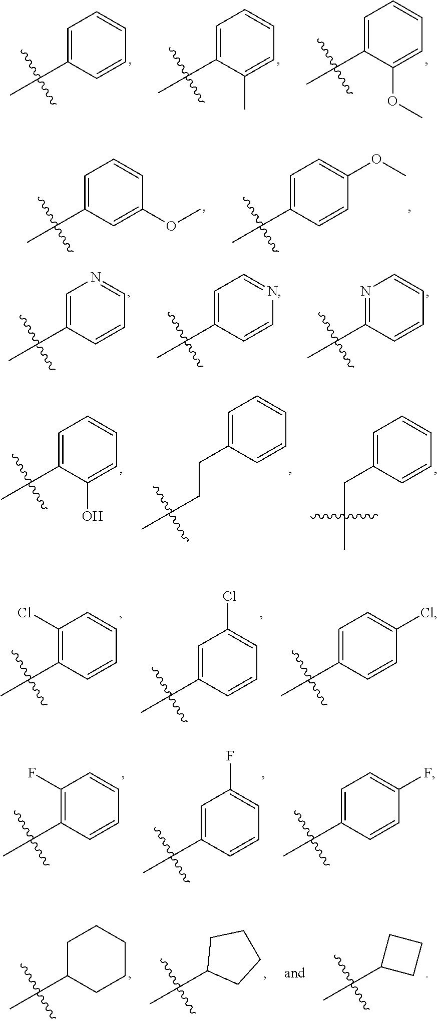
Y is a linker, and optionally an alkyl, alkenyl, heteroalkyl, or heteroalkenyl, where the hetero atom is S, N, or O that is optionally substituted;
R₃ is H, C₀-C₈ alkyl, C₀-C₂-alkyl-aryl or C₀-C₂-alkyl-heteroaryl, wherein the alkyls are independently substituted or non-substituted; and
R₄ is H, C₁-C₆ alkyl or cycloalkyl, optionally substituted, wherein one or more carbons may be replaced by oxygen, and wherein the alkyls are independently substituted or non-substituted, such as a structure of one of the following:

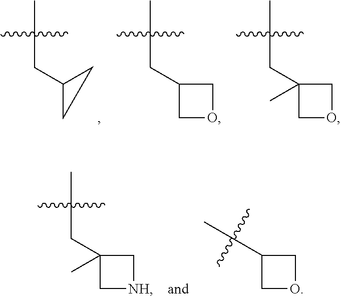

25-30. (canceled)

31. The method of claim 17, wherein the ALDHi is selected from one of the following, or a pharmaceutically acceptable salt thereof: 6-((3-fluorobenzyl)thio)-5-(o-tolyl)-1H-pyrazolo[3,4-d]pyrimidin-4(5H)-one, 6-((3-fluorobenzyl)thio)-5-phenyl-1H-pyrazolo[3,4-d]pyrimidin-4(5H)-one, 6-((3-fluorobenzyl)thio)-1-methyl-5-(o-tolyl)-1H-pyrazolo[3,4-d]pyrimidin-4(5H)-one, 6-((3-fluorobenzyl)thio)-2-methyl-5-(o-tolyl)-2H-pyrazolo[3,4-d]pyrimidin-4(5H)-one, 1-ethyl-6-((3-fluorobenzyl)thio)-5-(o-tolyl)-1H-pyrazolo[3,4-d]pyrimidin-4(5H)-one, 2-ethyl-6-((3-fluorobenzyl)thio)-5-(o-tolyl)-2H-pyrazolo[3,4-d]pyrimidin-4(5H)-one, 1-(cyclopropylmethyl)-6-((3-fluorobenzyl)thio)-5-(o-tolyl)-1H-pyrazolo[3,4-d]pyrimidin-4(5H)-one, 2-(cyclopropylmethyl)-6-((3-fluorobenzyl)thio)-5-(o-tolyl)-2H-pyrazolol[3,4-d]pyrimidin-4(5H)-one, 6-((3-fluorobenzyl)thio)-1-(oxetan-3-yl)-5-(o-tolyl)-1H-pyrazolo[3,4-d]pyrimidin-4(5H)-one, 6-((3-fluorobenzyl)thio)-2-(oxetan-3-yl)-5-(o-tolyl)-2H-pyrazolo[3,4-d]pyrimidin-4(5H)-one, 6-((3-fluorobenzyl)thio)-1-(oxetan-3-ylmethyl)-5-(o-tolyl)-1H-pyrazolol[3,4-d]pyrimidin-4(5H)-one, 6-((3-fluorobenzyl)thio)-2-(oxetan-3-ylmethyl)-5-(o-tolyl)-2H-pyrazolo[3,4-d]pyrimidin-4(5H)-one, 6-((3-fluorobenzyl)thio)-2-methyl-5-phenyl-2H-pyrazolo[3,4-d]pyrimidin-4(5H)-one, 1-ethyl-6-((3-fluorobenzyl)thio)-5-phenyl-1H-pyrazolo[3,4-d]pyrimidin-4(5H)-one, 2-ethyl-6-((3-fluorobenzyl)thio)-5-phenyl-2H-pyrazolo[3,4-d]pyrimidin-4(5H)-one, 1-(cyclopropylmethyl)-6-((3-fluorobenzyl)thio)-5-phenyl-1H-pyrazolo[3,4-d]pyrimidin-4(5H)-one, 2-(cyclopropylmethyl)-6-((3-fluorobenzyl)thio)-5-phenyl-2H-pyrazolo[3,4-d]pyrimidin-4(5H)-one, 6-((3-fluorobenzyl)thio)-1-(oxetan-3-yl)-5-phenyl-1H-pyrazolo[3,4-d]pyrimidin-4(5H)-one, 6-((3-fluorobenzyl)thio)-2-(oxetan-3-yl)-5-phenyl-2H-pyrazolo[3,4-d]pyrimidin-4(5H)-one, 6-((3-fluorobenzyl)thio)-1-(oxetan-3-ylmethyl)-5-phenyl-1H-pyrazolo[3,4-d]pyrimidin-4(5H)-one, 6-((3-fluorobenzyl)thio)-2-(oxetan-3-ylmethyl)-5-phenyl-2H-pyrazolo[3,4-d]pyrimidin-4(5H)-one, 6-((3-fluorobenzyl)amino)-5-(o-tolyl)-1H-pyrazolo[3,4-d]pyrimidin-4(5H)-one, 6-((3-fluorophenethyl)amino)-1-methyl-5-(o-tolyl)-1H-pyrazolo[3,4-d]pyrimidin-4(5H)-one, 6-((3-fluorobenzyl)oxy)-1-methyl-5-(o-tolyl)-1H-pyrazolo[3,4-d]pyrimidin-4(5H)-one, 6-((3-fluorophenoxy)methyl)-1-methyl-5-phenyl-1H-pyrazolo[3,4-d]pyrimidin-4(5H)-one, 6-(((3-fluorobenzyl)amino)methyl)-1-methyl-5-phenyl-1H-pyrazolo[3,4-d]pyrimidin-4(5H)-one, 6-((3-fluorobenzyl)thio)-1-methyl-5-phenyl-1H-pyrazolo[3,4-d]pyrimidin-4(5H)-one, 6-((3-fluorobenzyl)thio)-5-(2-methoxyphenyl)-1-methyl-1H-pyrazolo[3,4-d]pyrimidin-4(5H)-one, 6-((3-fluorobenzyl)thio)-5-(3-methoxyphenyl)-1-methyl-1H-pyrazolo[3,4-d]pyrimidin-4(5H)-one, 6-((3-fluorobenzyl)thio)-5-(4-methoxyphenyl)-1-methyl-1H-pyrazolo[3,4-d]pyrimidin-4(5H)-one 6-((3-fluorobenzyl)thio)-1-methyl-5-(pyridin-3-yl)-1H-pyrazolo[3,4-d]pyrimidin-4(5H)-one, 6-((3-fluorobenzyl)thio)-1-methyl-5-(pyridin-4-yl)-1H-pyrazolo[3,4-d]pyrimidin-4(5H)-one, 6-((3-fluorobenzyl)thio)-5-(2-hydroxyphenyl)-1-methyl-1H-pyrazolo[3,4-d]pyrimidin-4(5H)-one, 6-((3-fluorobenzyl)thio)-1-methyl-5-(pyridin-2-yl)-1H-pyrazolo[3,4-d]pyrimidin-4(5H)-one, 6-((3-fluorobenzyl)thio)-1-methyl-5-phenethyl-1H-pyrazolo[3,4-d]pyrimidin-4(5H)-one, 5-benzyl-6-((3-fluorobenzyl)thio)-1-methyl-1H-pyrazolo[3,4-d]pyrimidin-4(5H)-one, 6-((3-fluorobenzyl)thio)-1,5-dimethyl-1H-pyrazolo[3,4-d]pyrimidin-4(5H)-one, 6-(isopentylthio)-1-methyl-5-phenyl-1H-pyrazolo[3,4-d]pyrimidin-4(5H)-one, 6-((3-fluorobenzyl)thio)-3-methyl-5-(o-tolyl)-2H-pyrazolo[3,4-d]pyrimidin-4(5H)-one, 6-(benzo[b]thiophen-2-ylmethoxy)-1-methyl-5-(o-tolyl)-1H-pyrazolo[3,4-d]pyrimidin-4(5H)-one, 5-(2-chlorophenyl)-6-(3-fluorobenzylthio)-1-methyl-1H-pyrazolo[3,4-d]pyrimidin-4(5H)-one, 5-(2-chlorophenyl)-6-((3-fluorobenzyl)thio)-1-methyl-1H-pyrazolo[3,4-d]pyrimidin-4(5H)-one, 5-(3-chlorophenyl)-6-((3-fluorobenzyl)thio)-1-methyl-1H-pyrazolo[3,4-d]pyrimidin-4(5H)-one, 5-(4-chlorophenyl)-6-((3-fluorobenzyl)thio)-1-methyl-1H-pyrazolo[3,4-d]pyrimidin-4(5H)-one, 6-((3-fluorobenzyl)thio)-5-(2-fluorophenyl)-1-methyl-1H-pyrazolo[3,4-d]pyrimidin-4(5H)-one, 5-cyclohexyl-6-((3-fluorobenzyl)thio)-1-methyl-1H-pyrazolo[3,4-d]pyrimidin-4(5H)-one, 5-cyclopentyl-6-((3-fluorobenzyl)thio)-1-methyl-1H-pyrazolo[3,4-d]pyrimidin-4(5H)-one, 5-cyclobutyl-6-((3-fluorobenzyl)thio)-1-methyl-1H-pyrazolo[3,4-d]pyrimidin-4(5H)-one, (E)-6-(3-fluorostyryl)-1-methyl-5-phenyl-1H-pyrazolo[3,4-d]pyrimidin-4(5H)-one, 6-(3-fluorophenethyl)-1-methyl-5-phenyl-1H-pyrazolo[3,4-d]pyrimidin-4(5H)-one, 6-(((3-fluorophenyl)amino)methyl)-1-methyl-5-phenyl-1H-pyrazolo[3,4-d]pyrimidin-4(5H)-one, 6-(((3-fluorobenzyl)oxy)methyl)-1-methyl-5-phenyl-1H-pyrazolo[3,4-d]pyrimidin-4(5H)-one, 6-(((3-fluorophenyl)thio)methyl)-1-methyl-5-phenyl-1H-pyrazolo[3,4-d]pyrimidin-4(5H)-one, 1-methyl-6-((2-oxo-2-phenylethyl)thio)-5-phenyl-1H-pyrazolo[3,4-d]pyrimidin-4(5H)-one, 3-(((1-methyl-4-oxo-5-phenyl-4,5-dihydro-1H-pyrazolo[3,4-d]pyrimidin-6-yl)thio)methyl)benzonitrile, 6-((3-methoxybenzyl)thio)-1-methyl-5-phenyl-1H-pyrazolo[3,4-d]pyrimidin-4(5H)-one, 6-(benzylthio)-1-methyl-5-phenyl-1H-pyrazolo[3,4-d]pyrimidin-4(5H)-one, 1-methyl-5-phenyl-6-((3-(2,2,2-trifluoroacetyl)benzyl)thio)-1H-pyrazolo[3,4-d]pyrimidin-4(5H)-one, 3-(((1-methyl-4-oxo-5-phenyl-4,5-dihydro-1H-pyrazolo[3,4-d]pyrimidin-6-yl)thio)methyl)benzaldehyde, 4-(((1-methyl-4-oxo-5-phenyl-4,5-dihydro-1H-pyrazolo[3,4-d]pyrimidin-6-yl)thio)methyl)benzaldehyde, 6-((3-fluorobenzyl)oxy)-1-methyl-5-phenyl-1H-pyrazolo[3,4-d]pyrimidin-4(5H)-one, 1-methyl-5-phenyl-6-((1-phenylethyl)thio)-1H-pyrazolo[3,4-d]pyrimidin-4(5H)-one, 6-(((3-fluorophenyl)(methyl)amino)methyl)-1-methyl-5-phenyl-1H-pyrazolo[3,4-d]pyrimidin-4(5H)-one, 6-((3-fluorobenzyl)oxy)-2-(oxetan-3-ylmethyl)-5-phenyl-2H-pyrazolo[3,4-d]pyrimidin-4(5H)-one, 2-(oxetan-3-ylmethyl)-5-phenyl-6-((1-phenylethyl)thio)-2H-pyrazolo[3,4-d]pyrimidin-4(5H)-one, (R)-1-methyl-5-phenyl-6-((1-phenylethyl)thio)-1H-pyrazolo[3,4-d]pyrimidin-4(5H)-one, (S)-1-methyl-5-phenyl-6-((1-phenylethyl)thio)-1H-pyrazolo[3,4-d]pyrimidin-4(5H)-one, 1-methyl-5-phenyl-6-(1-phenylethoxy)-1H-pyrazolo[3,4-d]pyrimidin-4(5H)-one, 6-((3-fluorobenzyl)thio)-2-((3-methyloxetan-3-yl)methyl)-5-phenyl-2H-pyrazolo[3,4-d]pyrimidin-4(5H)-one, 3-((6-((3-fluorobenzyl)thio)-4-oxo-5-phenyl-4,5-dihydro-2H-pyrazolo[3,4-d]pyrimidin-2-yl)methyl)azetidin-1-ium sulfate, 1-methyl-5-phenyl-6-((1-phenylpropyl)thio)-1H-pyrazolo[3,4-d]pyrimidin-4(5H)-one, 6-((1-(3-fluorophenyl)ethyl)thio)-1-methyl-5-phenyl-1H-pyrazolo[3,4-d]pyrimidin-4(5H)-one, 6-(cyclohexylthio)-1-methyl-5-phenyl-1H-pyrazolo[3,4-d]pyrimidin-4(5H)-one, 1-(oxetan-3-yl)-5-phenyl-6-((1-phenylethyl)thio)-1H-pyrazolo[3,4-d]pyrimidin-4(5H)-one, 2-(oxetan-3-yl)-5-phenyl-6-((1-phenylethyl)thio)-2H-pyrazolo[3,4-d]pyrimidin-4(5H)-one, 1-methyl-6-((2-oxocyclopentyl)thio)-5-phenyl-1H-pyrazolo[3,4-d]pyrimidin-4(5H)-one, 1-methyl-6-((2-oxocyclohexyl)thio)-5-phenyl-1H-pyrazolo[3,4-d]pyrimidin-4(5H)-one, 1-methyl-5-phenyl-6-((2-phenylpropan-2-yl)thio)-1H-pyrazolo[3,4-d]pyrimidin-4(5H)-one, 6-((1-(3-hydroxyphenyl)ethyl)thio)-1-methyl-5-phenyl-1H-pyrazolo[3,4-d]pyrimidin-4(5H)-one, 1-methyl-5-phenyl-6-((1-(pyridin-2-yl)ethyl)thio)-1H-pyrazolo[3,4-d]pyrimidin-4(5H)-one, and 1-methyl-5-phenyl-6-((1-(pyridin-3-yl)ethyl)thio)-1H-pyrazolo[3,4-d]pyrimidin-4(5H)-one:
has a structure selected from: 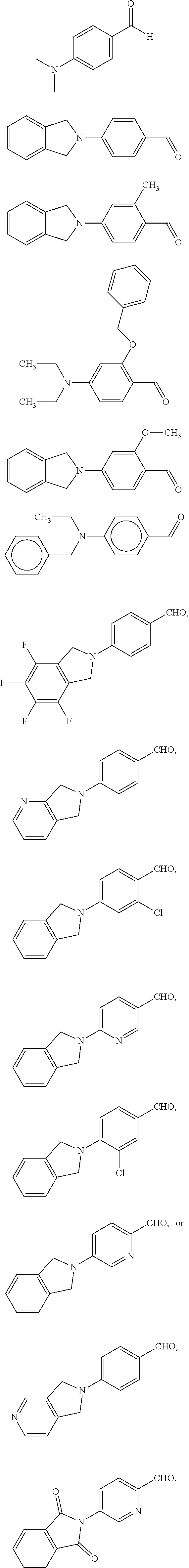
or a pharmaceutically acceptable salt thereof; or
has a structure 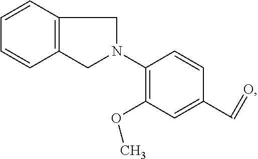 [SMILES: COc1cc(C=O)ccc1N1Cc2ccccc2C1]
or a pharmaceutically acceptable salt thereof.

32. (canceled)

33. The method of claim 17, wherein the ALDHi is selected from 673A, 257913 (913), 258085 (085), 262548 (548), 263052 (052), 263117 (117), 263118 (118), 263119 (119), 259122 (122), 263646 (646), 264202 (202), or a pharmaceutically acceptable salt thereof.

34. The method of claim 17, wherein the ALDHi is 673A, or a pharmaceutically acceptable salt thereof.

35. The method of claim 17, wherein the ALDHi has the structure: 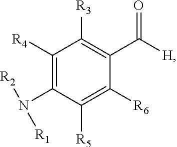
wherein R₁ and R₂ are independently or together, H, an alkyl, a cycloalkyl, together form a cycloalkyl or heterocyclic alkyl or aryl, an aryl, an alkenyl, a cycloalkyl, alkynl, or a substituted version of the aforementioned groups and R₃-R₆ are independently a halogen, H, an alkyl, a cycloalkyl, an aryl, an alkenyl, a cycloalkyl, alkynl, or a substituted version of the aforementioned groups, or a pharmaceutically acceptable salt thereof.

36-38. (canceled)

39. The method of claim 17, for treating cancer in a patient, comprising:
obtaining an antigen-presenting cell or a precursor thereof from the patient, wherein when an antigen-presenting cell precursor is obtained from the patient, further comprising culturing the precursor ex vivo in differentiation medium to differentiate the precursor to an antigen-presenting cell;
culturing the antigen-presenting cell in the presence of an antigen of a cancer cell from the patient to produce a matured antigen-presenting cell; and
administering the matured antigen-presenting cell to the patient with the ALDHi as an adjuvant therapy.

40. The method of claim 39, wherein the antigen-presenting cell or a precursor thereof is a dendritic cell or a precursor thereof, or a bone marrow progenitor cell, optionally obtained from the patient, and the method comprises differentiating the bone marrow progenitor cell to a dendritic cell.

41-42. (canceled)

43. The method of claim 17, wherein the ALDHi is administered to the patient on the same day as each instance of the immunotherapy (DO), and optionally for from one to ten days (D1-D10) after the immunotherapy.

44. (canceled)

45. The method of claim 17, wherein the patient has a tumor, and the ALDHi is administered in an amount effective to reduce tumor volume after 100 days in a patient, as compared to a tumor in a patient that is not treated with the ALDHi.

46-47. (canceled)
